(19) United States
(12) Patent Application Publication
(10) Pub. No.: US 2019/0367910 A1
(43) Pub. Date: Dec. 5, 2019

(54) METHODS AND COMPOSITIONS FOR RNA-GUIDED TREATMENT OF HIV INFECTION

(71) Applicant: TEMPLE UNIVERSITY - OF THE COMMONWEALTH SYSTEM OF HIGHER EDUCATION, Philadelphia, PA (US)

(72) Inventors: Kamel Khalili, Bala Cynwyd, PA (US); Wenhui Hu, Cherry Hill, NJ (US)

(21) Appl. No.: 15/764,119

(22) PCT Filed: Sep. 23, 2016
(86) PCT No.: PCT/US16/53413

§ 371 Date: Mar. 28, 2018

Related U.S. Application Data
(60) Provisional application No. 62/233,618, filed on Sep. 28, 2015.

(51) Int. Cl. C12N 15/11; C12N 9/22; A61K 35/17; C12N 15/90; A61P 31/18

(52) U.S. Cl. CPC C12N 15/11; C12N 9/22; A61K 35/17; C12N 2320/32; A61P 31/18; C12N 2800/80; C12N 2310/20; C12N 15/907

(57) ABSTRACT

The present disclosure provides compositions and methods for specific cleavage of target sequences in retroviruses, for example human immunodeficiency virus (HV-1). The compositions, which can include nucleic acids encoding a Clustered Regularly Interspace Short Palindromic Repeat (CRISPR) associated endonuclease and a guide RNA sequence complementary to a target sequence in a human immunodeficiency virus, can be delivered to the cells of a subject having or at risk for contracting an HV infection.

### STATEMENT REGARDING FEDERALLY SPONSORED RESEARCH

This invention was made with U.S. government support under grants awarded by the National Institutes of Health (NIH) to Kamel Khalili (P30MH092177), to Wenhui Hu (R01NS087971), and to Wenhui Hu and Kamel Khalili (R01 NS087971). The U.S. government may have certain rights in the invention.

### FIELD OF THE INVENTION

The present invention relates to compositions and methods for specific cleavage of target sequences in retroviruses, for example human immunodeficiency virus (HIV-1). The compositions, which can include nucleic acids encoding a Clustered Regularly Interspace Short Palindromic Repeat (CRISPR) associated endonuclease and a guide RNA sequence complementary to a target sequence in a human immunodeficiency virus, can be delivered to the cells of a subject having or at risk for contracting an HIV infection.

### BACKGROUND

AIDS remains a major public health problem, as over 35 million people worldwide are HIV-1-infected and new infections continue at steady rate of greater than two million per year. Antiretroviral therapy (ART) effectively controls viremia in virtually all, HIV-1 patients and partially restores the primary host cell (CD4⁺ T-cells), but fails to eliminate HIV-1 from latently-infected T-cells (Gandhi, et al., PLoS Med 7, e1000321(2010); Palella et al., N Engl J Med 338, 853-860 (1998)). In latently-infected CD4⁺ T cells, integrated proviral DNA copies persist in a dormant state, but can be reactivated to produce replication-competent virus when T-cells are activated, resulting in rapid viral rebound upon interruption of antiretroviral treatment (Chun, et al., Nature 387, 183-188 (1997); Chun, et al., Proc Natl Acad Sci USA 100, 1908-1913 (2003), Finzi, et al., Science 278, 1295-1300 (1997); Hermankova, et al., J Virol 77, 7388-7392 (2003); Siliciano, et al., Nat Med 9, 727-728 (2003); Wong, et al., Science 278, 1291-1295 (1997)). Therefore, most HIV-1-infected individuals, even those who respond very well to ART, must maintain life-long ART due to persistence of HIV-1-infected reservoir cells. During latency HIV infected cells produce little or no viral protein, thereby avoiding viral cytopathic effects and evading clearance by the host immune system. Because the resting CD4⁺ memory T-cell compartment (Bruner, et al., Trends Microbiol. 23, 192-203 (2015)) is thought to be the most prominent latently-infected cell pool, it is a key focus of research aimed at eradicating latent HIV-1 infection.

Recent efforts to eradicate HIV-1 from this cell population have used primarily a “shock and kill” approach, with the rationale that inducing HIV reactivation in CD4⁺ memory T may trigger elimination of virus-producing cells by cytolysis or host immune responses. For example, epigenetic modification of chromatin structure is critical for establishing viral reactivation. Consequently, inhibition of histone deacetylase (HDAC) by Trichostatin A (TSA) and vorinostat (SAHA) led to reactivation of latent virus in cell lines (Quivy, et al., J Virol 76, 11091-11093 (2002); Pearson, et al., J Virol 82, 12291-12303 (2008); Friedman, et al., J Virol 85, 9078-9089 (2011)). Accordingly, other HDACi, including vorinostat, valproic acid, panobinostat and rombidepsin have been tested ex vivo and have led, in the best cases, to transient increases in viremia (Archin, et al., Nature 487, 482-485 (2012); Blazkova, et al., J. Infect. Dis 206, 765-769 (2012)). Similarly, protein kinase C agonists, can potently reactivate HIV either singly or in combination with HDACi (Laird, et al., J Clin Invest, 125, 1901-1912 (2015); Bullen, et al., Nature Med 20:425-429 (2014)). However, there are multiple limitations of this approach: i) since a large fraction of HIV genomes in this reservoir are non-functional, not all integrated provirus can produce replication-competent virus (Ho, et al., Cell 155, 540-551 (2013)); ii) total numbers of CD4⁺ T cells reactivated from resting CD4⁺ T cell HIV-1 reservoirs, has been found by viral outgrowth assays to be much smaller than the numbers of cells infected, as detected by PCR-based assays, suggesting that not all cells within this reservoir are reactivated (Eriksson, et al., PLoS Pathog 9, el003174(2013)); iii) the cytotoxic T lymphocyte (CTL) immune response is not sufficiently robust to eliminate the reactivated infected cells (Shan, et al., Immunity 36, 491-501 (2012)) and iv) uninfected T-cells are not protected from HIV infection and can therefore sustain viral rebound.

Clustered, regularly interspaced, short palindromic repeats (CRISPR)-associated 9 (Cas9) nuclease systems have been shown to have wide utility in genome editing in a broad range of organisms including yeast, Drosophila, zebrafish, C. elegans, and mice, and has been heavily used by several laboratories in a broad range of in vivo and in vitro studies toward human diseases (Di Carlo et al., Nucl Acids Res 41:4336-4346 (2013); Gratz et al., Genetics 194, 1029-1035 (2013); Hwang et al., Nature Biotech 31, 227-229, (2013); Wang et al., 2013; Hu, et al., Proc Natl Acad Sci USA 111, 11461-11466 (2014)). In a CRISPR/Cas9 system, gene editing complexes are assembled. Each complex includes a Cas9 nuclease and a guide RNA (gRNA) complementary to a target sequence in a proviral DNA. The gRNA directs the Cas9 nuclease to engage and cleave the proviral DNA strand containing the target sequence. The Cas9/gRNA gene editing complex introduces one or more mutations into the viral DNA.

Recently, the CRISPR/Cas9 system has been modified to enable recognition of specific DNA sequences positioned within HIV-1 long terminal repeat (LTR) sequences (Hu, et al., Proc Natl Acad Sci USA 111, 11461-11466 (2014); Khalili et al., J Neurovirol 21, 310-321 (2015)). There is a need expand the existing repertoire of CRISPR/Cas9-mediated therapeutic capabilities, to include the capability of eradicating integrated HIV-1 DNA from latently infected patient T cells, and the capability of inducing resistance to HIV-1 infection in the T cells of patients at risk of infection.

### SUMMARY

A cure strategy for human immunodeficiency virus (HIV) infection includes methods that directly eliminate the proviral genome in HIV positive cells including CD4⁺ T-cells with limited, if any, harm to the host. In embodiment, the present invention provides compositions and methods for the treatment and prevention of retroviral infections, especially the human immunodeficiency virus, HIV-1. The compositions and methods utilize Cas9 and at least one gRNA, which form complexes that inactivate, and, in most cases eliminate, proviral HIV in the genomes of host T cells. In preferred embodiments, at least two gRNAs are included, with each gRNA directing a CRISPR-associated endonuclease to a different target site in an LTR of the HIV genome.

Specifically, the present invention provides Cas9/gRNA compositions for use in inactivating a proviral DNA integrated into the genome of a host cell latently infected with HIV. The present invention also provides a method of utilizing the Cas9/gRNA compositions to inactivate proviral HIV DNA in host cells.

The present invention further provides a lentiviral vector encoding Cas9 and at least one gRNA, for inactivating proviral DNA integrated into the genome of a host cell latently infected with HIV.

The present invention also provides an ex vivo method of eliminating a proviral DNA integrated into the genome of T cells latently infected with HIV. The method includes the steps of obtaining a population of host cells latently infected with HIV, such as the primary T cells of an AIDS patient; culturing the host cells ex vivo; treating the host cells with a Cas9 endonuclease, and at least one gRNA; and eliminating the proviral DNA from the host cell genome.

The present invention still further provides a method of treating a patient having latent HIVinfection of T cells. The method includes performing the steps of the ex vivo treatment method as previously stated; producing an HIV-eliminated T cell population; and returning the HIV-eliminated T cell population into the patient.

The present invention also provides a pharmaceutical Cas9/gRNA composition for inactivating integrated HIV DNA in the cells of a mammalian subject.

The present invention further provides a method of treating a mammalian subject infected with HIV, by administering an effective amount of the pharmaceutical composition as previously stated.

The present invention still further provides a method of prophyllaxis of HIV infection of T cells of a patient at risk of HIV infection. The method includes the step of establishing the stable expression of Cas9 and gRNA in patient T cells, either ex vivo or in vivo.

The present invention also provides kits for facilitating the application of the previously stated methods of treatment or prophylaxis of HIV infection.

### BRIEF DESCRIPTION OF THE DRAWINGS

FIGS. 1A-1D show that CRISPR/Cas9 eliminates HIV-1 expression in PMA/TSA treated, latently-infected human T-cell line. FIG. 1A: The top portion is a schematic representation of the structural organization of the integrated HIV-1 proviral DNA highlighting the position of the long terminal repeat (LTR), various viral genes spanned by the LTR, and the location of the reporter d2EGFP. The bottom portion of FIG. 1A is an illustration of the 5′ LTR and the nucleotide sequences of target regions A (gRNA A) and B (gRNA B) used for editing, and the motifs for binding of the various transcription factors. Arrow at +1 depicts the transcription start site. FIG. 1B is a gating diagram of EGFP flow cytometry and fluorescence microscopic imaging of the CD4⁺ T-cells before and after PMA/TSA treatment shows PMA/TSA-induced reactivation of latent virus in control cells expressing only Cas9, but not in cells expressing both Cas9 and gRNA. FIG. 1C: RT-PCR-based detection of gRNA A, gRNA B and β-actin RNA in cells transfected with plasmids expressing Cas9±gRNAs. β-actin is the RNA loading control. FIG. 1D: Detection of Cas9 protein by Western blot analysis in control cells and cells with ablated HIV-1/EGFP expression. β-tubulin served as the protein loading control.

FIGS. 2A-2D show the elimination of integrated HIV-1 DNA from the host T cell genome by Cas9/gRNAs targeting viral LTRs. FIG. 2A: DNA analysis shows 497- and 504-nucleotide amplicons detected, corresponding respectively to the HIV-1 LTRs in control cells and in cells co-expressing Cas9 and gRNAs. Positions of the amplicons corresponding to the RRE and β-actin are shown. FIG. 2B: Nucleotide composition of the amplified LTR DNA from CRISPR/Cas9-treated cells along with the positions of primers used for PCR amplification of the various regions of the viral genome. Integration of the 7-nucleotide InDel mutation after removal of the viral DNA fragment positioned between the B-motif of the 5′ and 3′ LTRs is shown. The seed sequence for gRNA B is highlighted in black. FIGS. 2C, 2D: The sites of HIV-1 integration in Chromosome 1 (FIG. 2C) and Chromosome 16 (FIG. 2D) are shown. In each figure, results of DNA analysis of the PCR product amplified by the specific primers (P1 and P2) derived from the cellular genes interrupted by viral DNA insertions are shown. Diagrams of each chromosome containing full-length integrated HIV-1 DNA before CRISPR/Cas9 treatment and the residual LTR DNA sequence after Cas9/gRNAs treatment are depicted, based on Sanger sequencing of the major DNA fragments seen on agarose gel. The asterisks in FIGS. 2C and 2D point to the minor DNA bands indicating the complete removal of viral DNA when either A or B targets within the 5′ or 3′ LTRs were used.

FIGS. 3A and 3F depict results from whole-genome sequencing which show excision of the entire provirus of two copies of HIV-1 by Cas9/gRNAs and gRNAs A and B in human T cells. FIGS. 3A, 3B: Integrative genomics view of the reads mapping against the HIV-1 genome (KM390026.1) called by BWA, revealed the presence of the HIV-1 proviral DNA sequence in the control cells with expression of Cas9 but not gRNAs (FIG. 3A) but their complete absence in T-cells after expression of both Cas9 and gRNAs A and B (FIG. 3B). FIGS. 3C, 3D: Structural variant CREST analysis identifies two breakpoints at the 5′ and 3′ ends of both LTRS supported by indicated reads after cleavage of Cas9/gRNAs A/B. Integrative genomics view (IGV) of the reads mapping against HIV-1 genome (KM390026.1) is illustrated. FIG. 3E: Identification of gRNAs (FIGS. 3A, 3B) specific breakpoint at 9389 site (red arrowhead) by structural variants called by CREST. The vertical purple line points to the position where the remaining of the 5′ and 3′ LTRs after cleavage were joined. FIG. 3F: Illustration of DNA sequence at the junction site (red arrowhead) after removal of the nucleotides between the precise cut sites, i.e. three nucleotides from PAM (red arrow) of the 5′ LTR at target A by gRNA A and the 3′ LTR at target B by gRNA B.

FIGS. 4A-4E show the impact of HIV-1-directed gene editing strategy on the host gene. FIG. 4A: Schematic presentation of Chromosome 1 highlighting the site of integration of HIV-1 proviral DNA in the cellular gene, RSBN1, and the position of neighboring genes. FIG. 4B: Expression of genes positioned at various proximities to the site of proviral integration before and after excision of the viral DNA by Cas9/gRNAs. Expression of the genes was identified by reverse transcription and qPCR, and the values were normalized to β-actin transcript. FIG. 4C: Linear structural organization of a segment of Chromosome 16 illustrating the position of MSRB1, the site of HIV-1 DNA integration and the nucleotide structure of exon 2 of MSRB1 where viral DNA is inserted. The position of several cellular genes near MSRB1 are shown. FIG. 4D: Results from SyberGreen qPCR illustrating expression of MSRB1 and it neighboring gene expression in cells prior to HIV-1 DNA eradication and after DNA eradication. The table shows target/reference for each cellular gene transcript obtained from 5 separate control and 5 separate HIV-1 eradicated single cell clones. FIG. 4E: Off-target evaluation by whole genome sequencing and bioinformatic interpretation. Graphic diagram demonstrates the position of predicted off-target sites with 3-7 nucleotide mismatches within the expanded 30, 300 and 600 bp flanking the filtered InDel sites in T-cells with excised HIV-1 DNA. The numbers beside the off-target sequence indicate the nucleotides of the 1200 bp expansion sequence. The mismatched nucleotides were highlighted in green in gRNA A off-target sites (blue) and orange in gRNA B off-target sites (purple). The PAM sequence was underlined with red. Of note, the off-target locations are far from the position of the InDels and exhibit no mutations at the predicted third nucleotide from PAM.

FIGS. 5A-5E: Lentivirus (LV) mediated Cas9/gRNA delivery suppresses HIV-1 infection in human T-cells. FIG. 5A: PCR fragment analysis of 2D10 T-cells treated with LV expressing gRNAs A/B, Cas9, or both Cas9 and gRNAs A/B. The positions of the full-length amplicon (417 bp) and the smaller DNA fragment (227 bp) after excision of the 190 bp between gRNAs A and B are shown. Amplification of the 270 bp β-actin DNA fragment is shown as a control. FIG. 5B: Representative scatter plots of GFP (HIV-1) and RFP (Cas9) expressing cells demonstrating that after LV infection 72.9% of 2D10 cells express Cas9, which after induction with PMA/TSA more than 45% of these cells (31.8%) show no evidence for GFP expression, indicative of HIV-1 DNA elimination. FIG. 5C: Experimental procedure layout of in vitro infection experiments in primary CD4⁺ T cells. CD4⁺ T cells were isolated from freshly prepared, antibody labeled PBMCs by negative selection on magnetic columns (Miltenyi Biotec) and then activated with 48 h anti-CD2/CD3/CD28 treatment followed by 6 days human rIL-2 mediated expansion. Next cells were infected with HIV-1 by spinoculation and 2 days later transduced with lentiviral cocktails containing lenti-Cas9 with or without lenti-gRNA LTR A/B. 4 days later cells supernatants and cells were harvested and analyzed for HIV-1 presence. FIG. 5D: CD4⁺ T-cells prepared from PBMC freshly isolated from buffy coat were infected with HIV-1_{JRFL} or HIV-1_{NL4-3} as described in Experimental Procedures, and HIV-1 copy number was determined by TaqMan qPCR and normalized to β-globin gene copy number. A significant reduction (48%) in the copy number of HIV-1_{JRFL} after 6 days of infection and even more dramatic decrease in HIV-1_{NL4-3} was observed upon LV-Cas9/gRNA expression in comparison to those that received LV-Cas9. FIG. 5E: PCR analysis of the LTR and β-actin DNA (control) from the HIV-1 infected CD4⁺ T-cells treated with LV-Cas9 in the presence or absence of LV-gRNAs A/B. The positions of the 398 bp HIV-1 LTR and 270 bp β-actin amplicons are shown.

FIGS. 6A-6F show the suppression of HIV-1 replication in the peripheral blood mononuclear cells (PBMCs) and CD4⁺ T-cells of HIV-1 infected patients. FIG. 6A: PBMCs from two HIV-1 infected volunteers were treated with LV-Cas9 or LV-Cas9 plus LV-gRNAs A/B (described in Materials and Methods) and viral DNA copies were determined by qPCR. As seen, a substantial decrease in the viral copy numbers was detected after normalization to 3-globin DNA. FIG. 6B: CD4⁺ T-cells isolated from PBMCs were expanded in media containing human IL-2 (20 U/ml) and infected with LV-Cas9 or LV-Cas9 plus LV-gRNA A/B followed by determination of viral DNA copy number 4 days later by qPCR. Similar to the PBMCs, a drastic reduction in the copy number of HIV-1 DNA was observed in cells receiving LV-Cas9/gRNAs compared to LV-Cas9. FIG. 6C: CD4⁺ T-cells after treatment with lentivirus vector expressing Cas9 or Cas9 and gRNAs A/B were harvested and viral replication was determined by p24 Gag ELISA. FIG. 6D: PCR analysis of DNAs isolated from the patient samples after lentivirus treatment by primers expanding −374/+43. The position of the 417 expected amplicons is shown. Control represent amplification of LTR DNA from PBMCs HIV-1_{JRFL} infected at 6 days of infection. FIG. 6E: PCR amplification of viral LTR (as described in FIG. 6D) using a different set of primers spanning −416-−19 of the LTR. The position of 398 bp amplicon is shown. FIG. 6F: TA cloning and sequencing of the LTR fragment (shown in FIG. 6E) from patient 2 showed insertion, deletion and single nucleotide variation (SNV) in some of the amplified DNA. Note that the assay cannot detect large DNA elimination that requires primers derived from the outside of the virus genome, i.e. flanking the site of integration.

FIG. 7A show the flow cytometry evaluation of several 2D10 clones transfected with plasmids expressing either Cas9 or Cas9 plus gRNAs. Treatment of the +Cas9/−gRNAs cells with PMA/TSA induced HIV-1 expression (GFP⁺) in 71%-89% of the cells. Conversely, cells transfected with Cas9 and gRNAs showed no significant response (1%-3%) to the treatment. FIG. 7B shows results from an RT-PCR assay for detection of gRNAs A and B in several clonal 2D10 cells after eradication of their latent HIV-1 genome. β-actin mRNA levels served as a control for the integrity of RNA preparation and loading. C11 represents RT-PCR of control (+Cas9/−gRNA) cells.

FIGS. 8A, 8B show the whole-genome sequencing and bioinformatic analysis of human T cells harboring integrated copies of HIV-1 proviral DNA. FIG. 8A: Details of the HIV-1 integration sites at the nucleotide levels on Chromosomes 1 and 16 are shown on the right. The host chromosomal DNA sequences are shown in red and the integrated DNA sequences are shown in black. Four deleted nucleotides (TAAG) are underlined in green. Four inter-chromosomal translocations (CTX) associated with HIV-1 are identified based on CREST calling of structural variants. FIG. 8B: Graphic representation of chromosomes 1 and 16, analyzed by NCBI, BLASTIN, highlights the correspondence between the HIV-1 genome and host chromosomes. (LTR, long terminal repeats).

FIGS. 9A, 9B show the results from DNA sequencing of the portion of Chromosome 1 depicting regions within RSNB1 where HIV-1 DNA is integrated. FIG. 9A: The positions of PAM along with nucleotide sequences of the LTR corresponding to gRNAs A and B (LTR A and B) are highlighted. FIG. 9B: DNA sequencing of PCR fragment showing the precise position of breakpoint and the seven nucleotide insertion at 3 nucleotides downstream from PAM.

FIG. 10A: DNA sequencing of host DNA in Chromosome 16 illustrating the precise sites of HIV-1 DNA integration within the MRSB1 gene and highlighted areas of InDel mutation. FIG. 10B: The positions of insertion of 8 nucleotides within the 5′-LTR after cleavage by gRNA A (at LTR A target) and insertion of 3 nucleotides upon the cleavage by gRNA B (at LTR B target) are shown.

FIGS. 11A, 11B show the results from an apoptotic assay which was used to assess the impact of Cas9/gRNA for eradication of HIV-1 on cell apoptosis. FIG. 11B is a bar graph showing the average results of the apoptotic assay performed on 14 T-cell clones infected only with Cas9 lentivirus and no gRNAs. For each sample the experiments were performed in triplicate, data are presented as average and standard deviation. The different colors represent the average percentage of cells detected in the different apoptotic stages, as shown in the table underneath the graph. The left panel of FIG. 11A shows the results for a representative sample. FIG. 11B is a bar graph which shows the results of the same apoptotic assay carried out on T-cell clones in which HIV-1 had been previously eradicated by infecting the cells with both Cas9 and gRNA lentiviruses. The left panel of FIG. 11B shows the results for a representative sample. The results show no significant differences between clones infected with Cas9 and eradicated ones, showing that gRNAs do not affect apoptotic cellular mechanisms.

FIGS. 12A, 12B show results from a cell viability assay which was used to investigate the impact of Cas9/gRNAs developed for HIV-1 eradication on cell viability. FIG. 12A is a bar graph showing the average results from the cell viability assay performed on 14 T-cell clones infected only with Cas9 lentivirus and no gRNAs. For each sample, the experiments were performed in triplicate, data are presented as average and standard deviation. The average percentage of live and dead cells is displayed respectively with blue and red. The left panel of FIG. 12A shows the results for a representative sample. FIG. 12B is a bar graph showing the results of the same cell viability assay carried out on T-cell clones in which HIV-1 had been previously eradicated by infecting the cells with both Cas9 and gRNA lentiviruses. The left panel of FIG. 12B shows the results for a representative sample. The results show no significant differences between clones infected with Cas9 and eradicated ones, showing that gRNA lentiviruses do not induce cell death.

FIGS. 13A, 13B show the results from a cell cycle asssay which was used to investigate the impact of Cas9/gRNAs developed for eradication of HIV-1 on cell cycle. FIG. 13B is a bar graph showing the average results of the cell cycle assay performed on 14 T-cell clones infected only with Cas9 lentivirus and no gRNAs. For each sample the experiments were performed in triplicate, data are presented as average and standard deviation. The average percentage of cells detected in the different cell cycle phases are displayed in different colors, as shown in the table underneath the bar graph. The left panel of FIG. 13A shows the results for a representative sample. FIG. 13B is a bar graph showing the results of the same cell cycle assay carried out on T-cell clones in which HIV-1 had been previously eradicated by infecting the cells with both Cas9 and gRNA lentiviruses. The left panel of FIG. 13B shows the results for a representative sample. The results show no significant differences between clones infected with Cas9 and eradicated ones, showing that gRNA lentiviruses do not affect cell cycle mechanisms.

FIG. 14 shows a graph showing the coverage depth (the left coordinate) and coverage rate (the right coordinate) of chromosome. The X-axis is chromosome number; the left Y-axis is the average depth of each chromosome, the right Y-axis is the fraction covered on each chromosome.

FIGS. 15A-15C show the protection of HIV-1 excised T-cell line from re-infection. FIG. 15A: Several latently infected T-cells after elimination of their HIV-1 genome were examined for expression of Cas9 (top panel) by Western blot and the presence of gRNA B (middle panel) and by RT-PCR. Expression of α-tubulin and β-actin serve as the loading controls for protein and RNA, respectively. FIG. 15B: T-cells with expression of Cas9 and/or gRNAs were infected with HIV-1 and at various times post-infection, the level of viral infection in each case was determined by flow cytometry. FIG. 15C shows the quantitative values of the experiment shown in FIG. 15B.

FIGS. 16A, 16B show results from patient derived primary PBMCs and CD4⁺ T-cell experiments. FIG. 16A: Blood samples from four HIV-1 positive patients on ART were obtained through the CNAC Basic Science Core 1 (Temple University, Philadelphia). AA: African-American, His: Hispanic. FIG. 16B: Schematic representation of experimental workflow for patient blood samples. CD4⁺ T-cells were isolated from freshly prepared, antibody labeled PBMCs by negative selection on magnetic columns (Miltenyi Biotec) and then activated with 48 hours anti-CD2/CD3/CD28 treatment followed by 6 days human rIL-2 mediated expansion. In parallel, PBMCs from the same blood samples were PHA-activated and similarly expanded with human rIL-2. Next, cells were transduced with lentiviral cocktails containing lenti-Cas9 with or without lenti-gRNA LTR A/B. 4 days later, supernatants and cells were harvested and analyzed for HIV-1 presence. FIG. 16C: The purity of CD4⁺ T-cells after isolation was checked by flow cytometry of FITC-conjugated anti-CD4 antibody labeled cells. Representative histograms of CD4 positive (GFP channel) cells after isolation in CD4 depleted and enriched populations.

FIG. 17 is a graph showing HIV-1 levels in patient derived PBMCs. p24 ELISA assay of PBMCs from Cases 3 and 4 after infection with lentivirus Cas9 or lentivirus Cas9 plus lentivirus gRNAs A and B. Cells were treated with anti-CD2, CD3, and CD28 covered beads (Miltenyi Biotec) at the cells:bead ratio of 2:21 or PMA/TSA cocktail (PMA 25 nM/TSA 250 nM) for 48 hours, then counted and Gag p24 in supernatants was measured.

FIGS. 18A-18B are amplification plots and standard curves used for absolute quantification of human beta-globin (FIGS. 18A, 18B) and HIV-1 Gag (FIGS. 18C, 18D) genes copy number in each sample. Serial dilutions of genomic DNA obtained from U1 monocytic cell line were prepared starting from 3.3 μg/ml which corresponds to 10⁵ genome copies in 10 l/reaction and finishing at 0.33 ng/ml corresponding to 10 genome copies in 10 μl/reaction. U1 cells contain 2 single, full length copies of HIV-1 provirus per genome, integrated in chromosome 2 and X, equal to beta-globin gene copies (2 per diploid genome).

### DETAILED DESCRIPTION

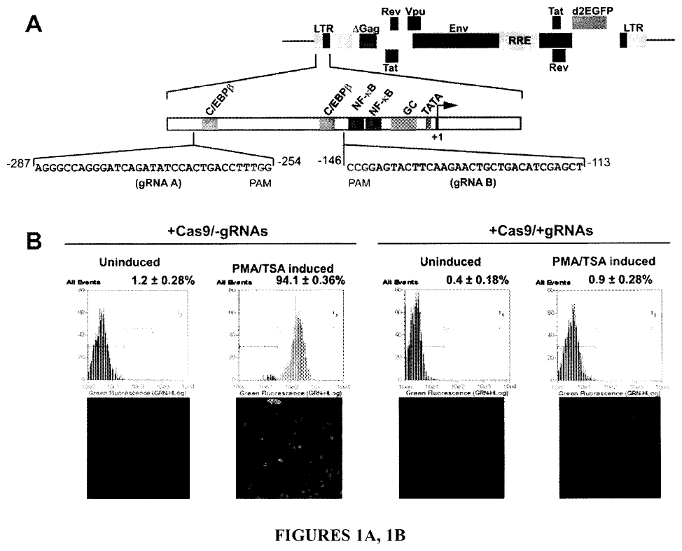

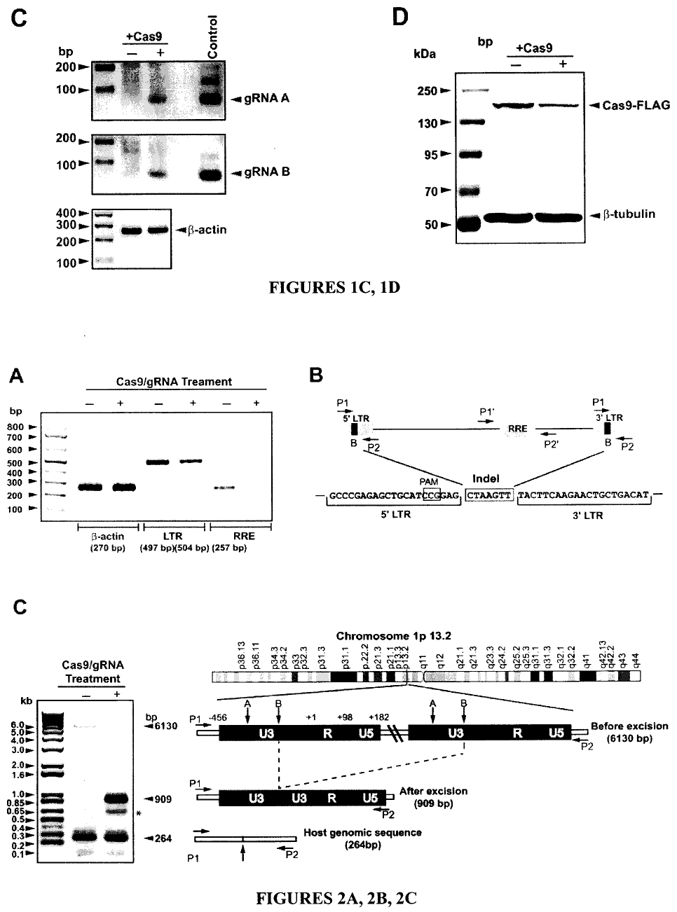

A CRISPR-Cas9 system according to the present invention includes at least one assembled gene editing complex comprising a CRISPR-associated nuclease, e.g., Cas9, and a guide RNA complementary to a target sequence situated on a strand of HIV proviral DNA that has integrated into a mammalian genome. Each gene editing complex can cleave the DNA within the target sequence, causing deletions and other mutations that inactivate proviral genome. In the preferred embodiments, the guide RNA is complementary to a target sequence occurring in each of the two LTR regions of the HIV provirus. In certain embodiments, the gRNAs are complimentary to sites in the U3 region of the LTR. In other embodiments, the gRNAs include gRNA A, which is complimentary to a target sequence in the region designated “gRNA A” in FIG. 1A, and gRNA B, which is complimentary to a target sequence in the region designated “gRNA B” in FIG. 1A. In a preferred embodiment, a combination of both gRNAs A and B in pairwise (“duplex”) fashion.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of“a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “eradication” of a retrovirus, e.g. human immunodeficiency virus (HIV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “percent sequence identity” or having “a sequence identity” refers to the degree of identity between any given query sequence and a subject sequence.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Compositions

The compositions disclosed herein may include nucleic acids encoding a CRISPR-associated endonuclease, such as Cas9. In some embodiments, one or more guide RNAs that are complementary to a target sequence of HIV may also be encoded. Accordingly, in some embodiments composition for use in inactivating a proviral DNA integrated into the genome of a host cell latently infected with human immunodeficiency virus (HIV), the composition comprises at least one isolated nucleic acid sequence encoding a Clustered Regularly Interspaced Short Palindromic Repeat (CRISPR)-associated endonuclease, and at least one guide RNA (gRNA), said at least one gRNA having a spacer sequence that is complementary to a target sequence in a long terminal repeat (LTR) of a proviral HIV DNA. In certain embodiments, the at least one gRNA comprises a nucleic acid sequence complementary to a target nucleic acid sequence having a sequence identity of at least 75% to one or more SEQ ID NOS: 1 to 66, fragments, mutants, variants or combinations thereof. In other embodiments, the at least one gRNA comprises at least one nucleic acid sequence complementary to a target nucleic acid sequence comprising SEQ ID NOS: 1 to 66, fragments, mutants, variants or combinations thereof. In certain embodiments, the at least one gRNA comprises a nucleic acid sequence having a sequence identity of at least 75% to one or more SEQ ID NOS: 1 to 66, fragments, mutants, variants or combinations thereof. In other embodiments, the at least one gRNA comprises at least one nucleic acid sequence comprising SEQ ID NOS: 1 to 66, fragments, mutants, variants or combinations thereof.

In yet other embodiments, the at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

The isolated nucleic acid can be encoded by a vector or encompassed in one or more delivery vehivles and formulations as described in detail below.

CRISPR-Associated Endonucleases:

The mechanism through which CRISPR/Cas9-induced mutations inactivate the provirus can vary. For example, the mutation can affect proviral replication, and viral gene expression. The mutation can comprise one or more deletions. The size of the deletion can vary from a single nucleotide base pair to about 10,000 base pairs. In some embodiments, the deletion can include all or substantially all of the proviral sequence. In some embodiments the deletion can eradicate the provirus. The mutation can also comprise one or more insertions, that is, the addition of one or more nucleotide base pairs to the proviral sequence. The size of the inserted sequence also may vary, for example from about one base pair to about 300 nucleotide base pairs. The mutation can comprise one or more point mutations, that is, the replacement of a single nucleotide with another nucleotide. Useful point mutations are those that have functional consequences, for example, mutations that result in the conversion of an amino acid codon into a termination codon, or that result in the production of a nonfunctional protein.

Three types (I-III) of CRISPR systems have been identified. CRISPR clusters contain spacers, the sequences complementary to antecedent mobile elements. CRISPR clusters are transcribed and processed into mature CRISPR RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The CRISPR-associated endonuclease can be a Cas9 nuclease. The Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Psuedomona aeruginosa, Escherichia coli, or other sequenced bacteria genomes and archaea, or other prokaryotic microogranisms may also be a source of the Cas9 sequence utilized in the embodiments disclosed herein.

The wild type Streptococcus pyogenes Cas9 sequence can be modified. An exemplary and preferred CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from another species, for example other Streptococcus species, such as Thermophilus; Psuedomona aeruginosa, Escherichia coli, or other sequenced bacteria genomes and archaea, or other prokaryotic microogranisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765, or Cas9 amino acid sequence of PX330 or PX260 (Addgene, Cambridge, Mass.).

The Cas9 nuclease sequence can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and Lcyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Guide RNA Sequences:

The compositions and methods of the present invention may include a sequence encoding a guide RNA that is complementary to a target sequence in HIV. The genetic variability of HIV is reflected in the multiple groups and subtypes that have been described. A collection of HIV sequences is compiled in the Los Alamos HIV databases and compendiums (i.e., the sequence database web site is hitp://www.hiv.lani.gov). The methods and compositions of the invention can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K. or group (for example, but not limited to any of the following Groups, N, O and P) of HIV.

The guide RNA can be a sequence complimentary to a coding or a non-coding sequence (i.e., a target sequence). For example, the guide RNA can be a sequence that is complementary to a HIV long terminal repeat (LTR) region.

Experiments disclosed in the Examples section show that the treatment of T lymphoid cells and primary human T cells with the Cas9 and gRNA compositions of the present invention causes the inactivation of integrated HIV-1 provirus, most commonly by eradication of the proviral genome. Results from whole genome sequencing and a comprehensive bioinformatic analysis ruled out any genotoxicity to normal host DNA.

Accordingly, the present invention encompasses a composition for use in inactivating a proviral DNA integrated into the genome of a host cell latently infected with a HIV. The composition includes at least one isolated nucleic acid sequence that encodes a CRISPR-associated endonuclease and at least one gRNA that is complementary to a target sequence in a long terminal repeat (LTR) of a proviral HIV DNA. The invention also encompasses a method of inactivating a proviral HIV DNA integrated into the genome of a host cell latently infected with HIV. The method includes the steps of treating the host cell with a composition including a CRISPR-associated endonuclease, and at least one gRNA complementary to a target sequence in a long terminal repeat (LTR) of a proviral HIV DNA. For both the composition and the method, the preferred gRNAs include gRNA A, gRNA B, or, most preferably, a combination of gRNA A and gRNA B.

A gRNA can include a mature crRNA that contains about 20 base pairs (bp) of unique targeting sequence, referred to as a “spacer”; and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (also known as a “protospacer”) on the target DNA. In the present invention, the crRNA and tracrRNA can be expressed separately or engineered into an artificial fusion gRNA via a synthetic stem loop (AGAAAU) to mimic the natural crRNA/tracrRNA duplex. Such gRNA can be synthesized or in vitro transcribed for direct RNA transfection or expressed from, for example, a U6 or H1-promoted RNA expression vector. When a gRNA is described as being complementary to a target DNA sequence, it will be understood that it is the spacer sequence of the gRNA that is actually complementary to the target DNA sequence.

Once guided to a target sequence by gRNA, Cas9 recognizes a trinucleotide (NGG) protospacer adjacent motif (PAM) to specify the cut site (the 3rd nucleotide from PAM).

The long terminal repeat (LTR) regions of HIV-1 are subdivided into U3, R and U5 regions. LTRs contain all of the required signals for gene expression, and are involved in the integration of a provirus into the genome of a host cell. For example, the basal or core promoter, a core enhancer and a modulatory region, are found within U3 while the transactivation response element is found within R. In HIV-1, the U5 region includes several sub-regions, for example, TAR or trans-acting responsive element, which is involved in transcriptional activation; Poly A, which is involved in dimerization and genome packaging; PBS or primer binding site; Psi or the packaging signal; DIS or dimer initiation site.

The preferred gRNAs of the present invention are each complementary to target sequences in the U3 region of the HIV-1 LTR. A gRNA A can be any gRNA complementary to either of two target sequences:

(SEQ ID NO: 1)
AGGGCCAGGGATCAGATATCCACTGACCTT; or
(SEQ ID NO: 2)
ATCAGATATCCACTGACCTT.

A gRNA B can be any gRNA complementary to either of two target sequences:

(SEQ ID NO: 3)
AGCTCGATGTCAGCAGTTCTTGAAGTACTC; or
(SEQ ID NO: 4)
CAGCAGTTCTTGAAGTACTC.

SEQ ID NOS: 1 and 3 are 30 bp gRNAs, which were employed in experiments described in detail in the examples section, wherein stable expression of gRNAs in lymphocytic host cells was achieved. SEQ ID NOS:2 and 4 are truncated 20 bp gRNAs, which were used in the construction of lentiviral vectors. The gRNAs of the present invention can also include a PAM sequence from the HIV-1 LTR at one end, although PAM sequences are not included in the gRNAs reported in the Examples. An exemplary gRNA A including a PAM sequence is AGGGCCAGGGATCAGATATCCACTGACCTTTGG (SEQ ID NO: 5). An exemplary gRNA B including a PAM sequence is AGCTCGATGTCAGCAGTTCTTGAAGTACTCCGG (SEQ ID NO: 6).

The gRNA sequences according to the present invention can be complementary to either the sense or anti-sense strands of the target sequences. They can include additional 5′ and/or 3′ sequences that may or may not be complementary to a target sequence. They can have less than 100% complementarity to a target sequence, for example 75% complementarity. The gRNA sequences can be employed as a combination of one or more different sequences, e.g., a multiplex configuration. Multiplex configurations can include combinations of two, three, four, five, six, seven, eight, nine, ten, or more different guide RNAs. In experiments disclosed in Examples 1 and 2, a duplex “two cut” strategy, employing both gRNA A and gRNA B, was found to be especially effective at producing viral inactivation and the eradication of sequences between the cleavages induced by Cas9 in each of the two LTRs of HIV-1.

Modified or Mutated Nucleic Acid Sequences: In some embodiments, any of the nucleic acid sequences may be modified or derived from a native nucleic acid sequence, for example, by introduction of mutations, deletions, substitutions, modification ofnucleobases, backbones and the like. The nucleic acid sequences include the vectors, gene-editing agents, gRNAs, etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH,—N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N. Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶⁻threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶⁻hydroxnorvalylcarbamoyl adenosine; 2′-O-ribosyladenosine (phosphate); inosine; 2′O-methyl inosine; 1-methyl inosine; 1,2′-O-dimethyl inosine; 2′-O-methyl guanosine; 1-methyl guanosine; N2-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N², 7-dimethyl guanosine; N², N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (pir.georgetown.edu), followed by analysis with the “short nearly identical sequences” Basic Local Alignment Search Tool (BLAST) algorithm on the NCBI website (ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

Recombinant Constructs and Delivery Vehicles:

Exemplary expression vectors for inclusion in the pharmaceutical composition include plasmid vectors and lentiviral vectors, but the present invention is not limited to these vectors. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus. The vector can also include origins of replication, scaffold attachment regions (SARs), regulatory regions and the like. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns. The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

In experiments disclosed in the Examples section, lentiviral vectors were found to be effective at achieving expression of the Cas9 and gRNAs of the present invention in human T lymphocyte lines and, for the first time, in primary cultures of human T cells, including T cells derived from HIV-1⁺ patients. In the primary T cells from HIV⁺ patients, combined expression of lentivirally delivered Cas9 and gRNAs A and B significantly reduced viral copy number and viral protein expression. This represents a critical advance in the therapy of HIV⁺ patients over the prior gene editing art.

Therefore, the present invention encompasses a lentiviral vector composition for inactivating proviral DNA integrated into the genome of a host cell latently infected with HIV. The composition includes an isolated nucleic acid encoding a CRISPR-associated endonuclease, and at least one isolated nucleic acid encoding at least one guide gRNA including a spacer sequence that is complementary to a target sequence in an LTR of a proviral HIV DNA, with the isolated nucleic acids being included in at least one lentiviral expression vector. The lentiviral expression vector induces the expression of the CRISPR-associated endonuclease and the at least one gRNA in a host cell.

All of the isolated nucleic acids can be included in a single lentiviral expression vector, or the nucleic acids can be subdivided into any suitable combination of lentiviral vectors. For example, the CRISPR associated endonuclease can be incorporated into a first lentiviral expression vector, a first gRNA can be incorporated into a second lentiviral expression vector, and a second gRNA can be incorporated into a third lentiviral expression vector. When multiple expression vectors are used, it is not necessary all of them be lentiviral vectors.

The results of Example 2 also demonstrate the utility of exposing latently infected T cells in ex vivo culture to the Cas9 and gRNA compositions of the present invention. Combinations of gRNA A and gRNA B were found to yield optimal eradication of integrated HIV proviral DNA. One use for this capability is an adoptive therapy, entailing the ex vivo culture of a patient's HIV infected cells with the compositions of the present invention, and the return of the HIV-eliminated cells to the patient.

Recombinant constructs are also provided herein and can be used to transform cells. A recombinant nucleic acid construct comprises a nucleic acid encoding a Cas9 and/or a guide RNA complementary to a target sequence in HIV as described herein, operably linked to a regulatory region suitable for expressing the Cas9 and/or a guide RNA complementary to a target sequence in HIV in the cell. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for Cas9 can be modified such that optimal expression in a particular organism is obtained, using appropriate codon bias tables for that organism.

Several delivery methods may be utilized in conjunction with the molecules embodied herein for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005).

Vectors for the in vitro or in vivo expression of any of the polynucleotides embodied herein include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors is known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991). In some embodiments the vector is a replication defective vector. Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

In some embodiments, the vector is a single stranded DNA producing vectors which can produce the expressed products intracellularly. See for example, Chen et al, BioTechniques, 34: 167-171 (2003), which is incorporated herein, by reference, in its entirety.

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infections. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding CRISPR/Cas and/or a guide RNA complementary to a target sequence of HIV, as described above.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present invention can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, aLangmuir 21(20):9183-6, incorporated by reference.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more CRISPR/Cas vectors and gRNAs. In some embodiments, the methods of the invention can be applied ex vivo. That is, a subject's cells can be removed from the body and treated with the compositions in culture to excise, for example, HIV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Therefore, the present invention encompasses a method of eliminating a proviral DNA integrated into the genome of ex vivo cultured host cells latently infected with HIV, wherein a proviral HIV DNA is integrated into the host cell genome. The method includes the steps of obtaining a population of host cells latently infected with HIV; culturing the host cells ex vivo; treating the host cells with a composition including a CRISPR-associated endonuclease, and at least one gRNA complementary to a target sequence in an LTR of the proviral HIV DNA; and eliminating the proviral DNA from the host cell genome. The same method steps are also useful for treating the donor of the latently infected host cell population when the following additional steps are added: producing an HIV-eliminated T cell population; infusing the HIV-eliminated T cell population into the patient; and treating the patient.

The previously stated lentiviral delivery system described in the Examples section is a preferred system for the ex vivo transduction of the CRISPR-associated endonuclease and the gRNAs in patient T cells or other latently infected host cells. Alternatively, any suitable expression vector system can be employed, including, but not limited to, those previously enumerated.

The compositions and methods that have proven effective for ex vivo treatment of latently infected T cells are very likely to be effective in vivo, if delivered by means of one or more suitable expression vectors. Therfore, the present invention encompasses a pharmaceutical composition for the inactivation of integrated HIV DNA in the cells of a mammalian subject, including an isolated nucleic acid sequence encoding a CRISPR-associated endonuclease, and at least one isolated nucleic acid sequence encoding at least one gRNA that is complementary to a target sequence in an LTR of a proviral HIV DNA. Preferably, a combination of gRNA A and gRNA B is included. It is also preferable that the pharmaceutical composition also include at least one expression vector in which the isolated nucleic acid sequences are encoded.

The present invention also encompasses a method of treating a mammalian subject infected with HIV, including the steps of: determining that a mammalian subject is infected with HIV, administering an effective amount of the previously stated pharmaceutical composition to the subject, and treating the subject for HIV infection.

Pharmaceutical compositions according to the present invention can be prepared in a variety of ways known to one of ordinary skill in the art. For example, the nucleic acids and vectors described above can be formulated in compositions for application to cells in tissue culture or for administration to a patient or subject. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein, in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating a nucleic acid encoding Cas9 or a variant Cas9 and at least one gRNA sequence complementary to a target HIV; or it can include a vector encoding these components. Alternatively, the compositions can be formulated as a nanoparticle encapsulating the CRISPR-associated endonuclease the polypeptides encoded by one or more of the nucleic acid compositions of the present invention.

In methods of treatment of HIV infection, a subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein.

The compositions of the present invention, when stably expressed in potential host cells, reduce or prevent new infection by HIV. Exemplary methods and results are disclosed in the Examples section. Accordingly, the present invention encompasses a method of preventing HIV infection of T cells of a patient at risk of HIV infection. The method includes the steps of determining that a patient is at risk of HIV infection; exposing T cells of the patient to an effective amount of an expression vector composition including an isolated nucleic acid encoding a CRISPR-associated endonuclease, and at least one isolated nucleic acid encoding at least one gRNA that is complementary to a target sequence in the an LTR of HIV DNA; stably expressing in the T cells the CRISPR-associated endonuclease and the at least one gRNA; and preventing HIV infection of the T cells.

A subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can also be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

The present invention also includes a kit to facilitate the application of the previously stated methods of treatment and prophylaxis of HIV infection. The kit includes a measured amount of a composition including at least one isolated nucleic acid sequence encoding a CRISPR-associated endonuclease, and at least one nucleic acid sequence encoding one or more gRNAs, wherein each of the gRNAs includes a spacer sequence complementary to a target sequence in a long terminal repeat (LTR) of an HIV provirus. The kit also includes and one or more items selected from the group consisting of packaging material, a package insert comprising instructions for use, a sterile fluid, a syringe and a sterile container. gRNAs A and B are the preferred gRNAs. In a preferred embodiment, the nucleic acid sequences are included in an expression vector, such as the lentiviral expression vector system described in detail in Example 1. The kit can also include a suitable stabilizer, a carrier molecule, a flavoring, or the like, as appropriate for the intended use.

### EXAMPLES

### Example 1: Materials and Methods

Cell Culture

1. Stable Cell Lines.

The Jurkat 2D10 reporter cell line has been described previously (Pearson, et al., J Virol 82, 12291-12303 (2008)) and was cultured in RPMI medium containing 10% FBS and gentamicin (10 μg/ml). 2×10⁶ cells were electroporated with 10 μg control pX260 plasmid or pX260 LTR-A and pX260 LTR-B plasmids, 5 g each (Neon System, Invitrogen, 3 times 10 ms/1350V impulse). 48 h later medium was replaced with medium containing puromycin 0.5 ug/ml. After a one week selection, puromycin was removed and cells were allowed to grow for another week. Next, cells were diluted to a concentration of 10 cells/ml and plated in 96 well plates, 50 l/well. After two weeks, single cell clones were screened for GFP tagged HIV-1 reporter reactivation (12 h PMA 25 nM/TSA 250 nM treatment) using a Guava EASYCYTE Mini flow cytometer. The non-reactive clones were used for further analysis.

2. Primary CD4⁺ Cell Isolation and Expansion.

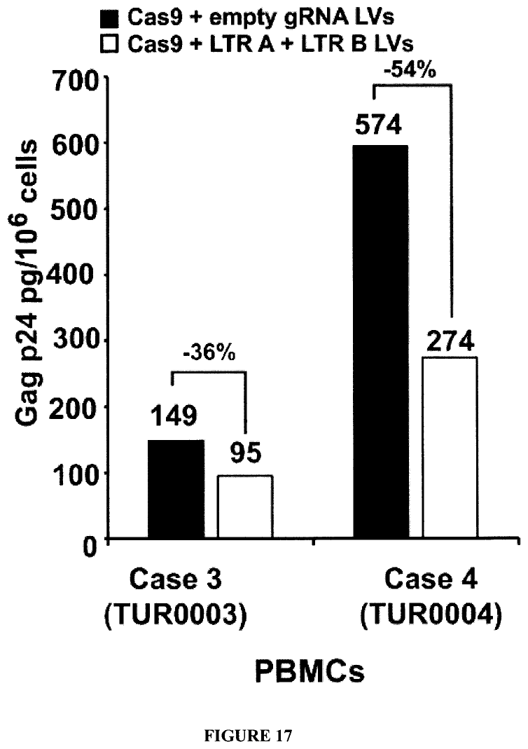

Buffy coat and patient blood samples were obtained through CNAC Basic Science Core I (Temple University School of Medicine, Philadelphia). PBMCs were isolated from human peripheral blood by density gradient centrifugation using Ficoll-Paque reagent. Blood/buffy coat samples volume was adjusted to 30 ml with HBSS buffer, gently layered on 15 ml of Ficoll-Paque cushion and centrifuged for 30 minutes at 1500 RPM. PBMCs containing layer was collected, washed 3 times in HBSS buffer and counted. Further isolation of CD4⁺ T-cells was performed using CD4⁺ T cell isolation kit human (Miltenyi Biotec). Cells (10⁷) were labeled with biotin-conjugated antibody cocktail (anti-CD8, CD14, CD15, CD16, CD19, CD36, CD56, CD123, CD235a, TCRy/6), then mixed with MicroBeads conjugated with anti-biotin and anti-CD61 antibodies and separated on MACS LS columns. Flow-through unlabeled cells representing the CD4⁺ enriched fraction was collected, and purity was confirmed by CD4-FITC FACS (94-97% CD4⁺ positive, see FIG. 17). Next, cells were expanded using T-cell activation/expansion kit according to the manufacturer's protocol (Miltenyi Biotec). Briefly, 2.5×10⁶ cells/ml were mixed with anti-CD2, CD3, CD28 antibody-coated MicroBeads in ratio of cells:beads of 2:1. After 2 days, cells were gently pipetted to disrupt clumps and one volume of fresh growth medium containing human rIL-2 was added. Medium was replaced every 3 days. All primary cells were grown in RPMI with 10% FBS and gentamicin (10 μg/ml) supplemented with human rIL-2 at concentration of 20 U/ml (NIH AIDS Reagent Program, Division of AIDS, NIAID, NIH: Human rIL-2 from Dr. Maurice Gately (Hoffmann-La Roche Inc.). All procedures involving AIDS patient samples and in vitro infected cells were performed in a BL2⁺ lab.

Lentiviral Delivery

1. Cloning Lentiviral Constructs.

The “all-in-one” pX260-U6-DR-BB-DR-Cbh-NLS-hSpCas9-NLSH1-shorttracr-PGK-puro (Addgene 42229) vectors containing LTR target A and B were described previously (Hu, et al., Proc Natl Acad Sci USA 111, 11461-11466 (2014)). For lentiviral delivery into primary cells, DNA segments expressing gRNA for LTR target A and B were shortened to 20 nucleotides (Table I section 5) and first subcloned into U6-chimeric-gRNA expressing cassette of pX330-U6-Chimeric_BB-CBh-hSpCas9 (Addgene 42230). Then the whole gRNA expressing cassette was PCR amplified with Mlu1/BamH1 extended primers (T560/T561 see Table I section 5), digested, and inserted into Mlu1/BamH1 sites of pKLV-U6gRNA(Bbsl)-PGKpuro2ABFP (Addgene 50946).

2. Lentivirus Packaging and Purification.

The obtained pKLV-U6-LTR A/B-PGKpuro2ABFP were packaged into lentiviral particles by co-transfection of HEK293T cells with pMDLg/pRRE (Addgene 12251), pRSV-Rev (Addgene 12253) and pCMV-VSV-G (Addgene 8454). For packaging Cas9 into lentiviral particles following vectors were used: pCW-Cas9 (Addgene 50661), psPAX2 (Addgene 12260), and pCMV-VSV-G (Addgene 8454). For some experiments pLV-EF1a-Cas9v1-T2A-RFP lentivirus was used (Biosettia Inc.). HEK 293T cells were co-transfected using CaPO₄ precipitation method in the presence of chloroquine (50 M) with packaging lentiviral vectors mixtures at 30 μg total DNA/2.5×10⁶ cells/100 mm dish. The next day, the medium was replaced and at 24 and 48 h later supernatants were collected, clarified at 3000 RPM for 10 minutes, 0.45 μm filtered, and concentrated by ultracentrifugation (2 h, 25000 RPMI, with 20% sucrose cushion). Lentiviral pellets were resuspended in HBSS by gentle agitation overnight, aliquoted, and tittered in HEK 293T cells. pCW-Cas9 lentivirus was tittered by FLAG immunocytochemistry, pKLV-U6-LTR A/B-PGKpuro2ABFP lentiviruses by BFP fluorescent microscopy.

3. Lentiviral Transduction of Primary Cells.

24 h before transduction, growth medium was replaced, and cells were activated by incubation with anti-CD2/CD3/CD28 antibody-coated magnetic beads (Miltenyi Biotec) at cells/beads ratio 2:1. Next day 2.5×10⁵ cells were infected with 12.5×10⁵ IU of pCW-Cas9 lentivirus, together with 25×10⁵ IU pKLV-empty lentivirus or 12.5×10⁵ IU of each pKLV-LTR target A and pKLV-LTR target B lentiviruses (total MOI 15). Cells were spinoculated for 2 h at 2700 RPM, 32° C. in 150 μl inoculum containing 8 μg/ml polybrene, then resuspended and left for 4 h, then 150 μl of growth medium was added. Next day cells were washed 3 times in 1 ml of PBS and incubated in growth medium containing human rIL-2 (20 U/ml).

Virus Assays and Detection

1. Viral Stocks.

HIV-1_{JRFL} crude stock was prepared from supernatants of PBMCs infected with HIV-1 for 6 days, clarified at 3000 RPM for 10 minutes and 0.45 μm filtered. HIV-1_{NL4-3}-EGFPP2A-Nef reporter virus was prepared by transfecting HEK 293T cells with pNL4-3-EGFP-P2A-Nef plasmid and processed as for lentiviral stocks (see above). HIV-1_{JRFL} was titered using Gag p24 ELISA, HIV-1_{NL43}-EGFP-P2A-Nef by GFP-FACS of infected HEK 293T cells.

2. In Vitro HIV-1 Infection.

CD4⁺ T-cells prepared from primary PBMCs were activated and expanded for one week before HIV-1 infection. Infection was done using crude HIV-1 stocks at 300 ng of Gag p24/10⁶ cells/I ml by spinoculation for 2 h at 2700 RPM, 32° C. in serum free medium containing 8 g/ml polybrene, then resuspended and left for 4 h followed by washing 3 times in PBS, and finally incubated in growth medium containing human rIL-2 (20 U/ml). In the case of CD4⁺ T cells infection, cells were activated and expanded for one week before HIV-1 infection. Jurkat 2D10 cells were reinfected without spinoculation by simple overnight incubation of the cells with diluted viral stock in the presence of polybrene 8 μg/ml.

3. HIV-1 DNA Detection and Quantification.

Genomic DNA was isolated from cells using a NUCLEOSPIN Tissue kit (Macherey-Nagel) according to the protocol of the manufacturer. For LTR specific PCRs (see Table I section 1), 100 ng of extracted DNA was subjected to PCR using FAIL SAFE PCR kit and buffer D (Epicentre) under the following PCR conditions: 98° C., 5 minutes, 30 cycles (98° C. 30 s, 55° C. 30 s, 72° C. 30 s), 72° C. 7 minutes and resolved in 2% agarose gel. Integration site specific PCRs (see Table I section 2) were performed on 250 ng of genomic DNA using a Long Range PCR kit (Qiagen) under the following conditions: 93° C. 3 minutes, 35 cycles (93° C. 15 s, 55° C. 30 s, 62° C. 7.5 minutes). PCR products were subjected to agarose gel electrophoresis, gel purified, cloned into TA vector (Invitrogen) and sent for Sanger sequencing (Genewiz). HIV-1 DNA was quantified using TAQMAN qPCR specific for HIV-1 Gag gene, and cellular beta-globin gene as a reference (see Table I, section 6.). Prior to qPCR, genomic DNA from infected cells was diluted to 10 ng/μl and then 5 μl (=50 ng) was taken per reaction/well. Reaction mixtures were prepared using Platinum Taq DNA Polymerase (Invitrogen) according to a simplified procedure from M. K. Liszewski et al., Methods, 47(4): 254-260 (2009). Standard was prepared from serial dilutions of U1 cells (NIH AIDS Reagent Program, Division of AIDS, NIAID, NIH: HIV-1 infected Cells (U1) from Dr. Thomas Folks, Folks, et al., Science 238, 800-802 (1987) genomic DNA, since it contains two single copies of HIV-1 provirus per diploid genome, equal to beta-globin gene copy number. qPCR conditions for Gag gene: 98 OC 5 minutes, 45 cycles (98° C. 15 s, 62° C. 30 s with acquisition); for beta-globin gene: 98° C. 5 minutes, 45 cycles (98° C. 15 s, 62° C. 30 s with acquisition, 72° C. 1 minute). Reactions were carried out and data analyzed in a LightCycler480 (Roche).

4. p24 ELISA.

Infection levels were quantified by subjecting supernatants from infected cells to p24 Gag antigen capture ELISA (ABL Inc.). For normalization, total cell number and supernatant volumes were recorded.

Host Genome Analysis

1. Genomic DNA Preparation, Whole Genome Sequencing and Bioinformatics Analysis.

The single subclone control C11 and experimental AB5 from parent 2D10 T cells were validated for target cut efficiency and functional suppression of HIV-1 EGFP reporter reactivation. The genomic DNA was isolated with NUCLEOSPIN Tissue kit (Macherey-Nagel) according to the protocol of the manufacturer. The genomic DNA was submitted to Novogene Bioinformatics Institute (novogene.com/en/) for WGS and bioinformatics analysis. Briefly, DNA quality was further verified on 1% agarose gels, DNA purity was checked using the NANOPHOTOMETER® spectrophotometer (IMPLEN, CA, USA), and DNA concentration was measured using QUBIT® DNA Assay Kit in QUBIT® 2.0 Flurometer (Life Technologies, CA, USA). A total amount of 1.5 μg DNA per sample was used for sequencing library generation using a Truseq Nano DNA HT Sample Preparation Kit (Illumina USA) following manufacturer's recommendations and index codes were added to attribute sequences to each sample. The DNA sample was fragmented by sonication to a size of 350 bp, then DNA fragments were end-polished, A-tailed, and ligated with the full-length adapter for Illumina sequencing, with further PCR amplification. Finally, PCR products were purified (AMPure XP system), and libraries were analyzed for size distribution by an Agilent2100 Bioanalyzer, and quantified using real-time PCR. The clustering of the index-coded samples was performed on a cBot Cluster Generation System using Hiseq X HD PE Cluster Kit (Illumina), according to the manufacturer's instructions. After cluster generation, the library preparations were sequenced on an Illunina Hiseq X Ten platform and paired-end reads were generated. The original raw data were transformed to sequenced reads by base calling and recorded in a FASTQ file, which contains sequence information (reads) and corresponding sequencing quality information. After filtering out any reads with adapter (>10 nucleotide aligned to the adaptor, allowing <10% mismatches), >10% unidentified nucleotides, >50% bases having phred quality <5, or putative PCR duplicates, a total of 342.67 Gb clean reads (average 109.25x coverage) for the control sample and 369.55 Gb (112.72x) for AB5 sample were retained for further assembly. Burrows-Wheeler Aligner (BWA) software (Li and Durbin, Bioinformatics 25, 1754-1760 (2009)) was utilized to map the paired end clean reads to the reference human genome (UCSC hgl9) and HIV-1 genome (KM390026.1). Then, Picard Samtools (Li H, et al. Bioinformatics 25, 2078-2079 (2009); broadinstitute.github.io/picard/), GATK (DePristo, Banks, et al. Nat Genetics 43, 491-498 (2011)) and Samtools (Li, Handsaker et al. Bioinformatics 25, 2078-2079 (2009)) were used to do duplicate removal, local realignment, and base quality recalibration to generate final BAM file for computation of the sequence coverage and depth. Candidate indels were filtered on several criteria using Python and the PyVCF (version 0.6.0), and PyFasta packages (version 0.5.0). The potential off-target effects of Cas9/LTR-gRNAs (AB5 group) on host genome were focused on by comparing the difference between the control (C11) and the experimental group (AB5). The SNP was detected by muTect (Cibulskis, Lawrence, et al. Nat Biotechnol 31, 213-219 (2013)), the indel was detected by Strelka (Saunders, Wong, et al., Bioinformatics 28, 1811-1817 (2012)) and the structural variants (SV) were detected by CREST (Wang, Mullighan, et al. Nat Methods 8, 652-654 (2011)). The total number of indels unique in the AB5 group was 32,399, and filtered by public database (dbSNP) (Sherry, Ward, et al. Nucl Acids Res 29, 308-311 (2001)) and heterozygous indels. Then, sequences were extracted from 300 bp (600 bp) upstream to 300 bp (600 bp) downstream of the indel sites as described previously (Hu, Kaminski, et al. Proc Natl Acad Sci USA 111, 11461-11466 (2014); Veres, Gosis, et al. Cell Stem Cell 15, 27-30 (2014)). Sequences were extracted from 300 bp (600 bp) upstream to 300 bp (600 bp) downstream of the indel sites and then compared to the predicted potential off-target sequence LTR-A/B+NRG. Similarly, SV analysis detected 42 deletions and 10 insertions in the AB5 group, and the extraction sequences at ±300 bp (600 bp) were compared against predicted off-target sequence LTR-A/B+NRG. To determine the integration site(s) of HIV-1, CREST (Wang, Mullighan, et al. Nat Methods 8, 652-654 (2011)) was used to detect the SV of the control sample that related to the HIV-1 genome.

2. Surveyor Assay.

The presence of mutations in PCR products from 6 predicted off-target sites (Table I, section 1.) was tested using a SURVEYOR Mutation Detection Kit (Transgenomic), according to the protocol of the manufacturer. Briefly, heterogeneous PCR product was denatured for 10 minutes at 95° C. and hybridized by gradual cooling using a thermocycler. Next 300 ng of hybridized DNA (9 μl) was subjected to digestion with 0.25 μl of SURVEYOR Nuclease in the presence of 0.25 μl SURVEYOR Enhancer S and 15 mM MgCl₂S for 4 h at 42° C. Then, Stop Solution was added and samples were resolved in 2% agarose gel together with undigested controls.

3. Reverse Transcription and PCR.

Total RNA was extracted from Jurkat cells using an RNeasy kit (Qiagen) with on column DNAse I digestion. Next, 0.5 μg of RNA was used for M-MLV reverse transcription reactions (Invitrogen). For gRNA expression screening, specific reverse primer (pX260-crRNA-3′/R, Table I, section 3) was used in RT reaction followed by standard PCR using target A or B sense oligos as forward primers (Table I, section 5) and agarose gel electrophoresis. For checking neighboring genes, expression oligo-dT primer mix was used in RT, and cDNA was subjected to SYBERGREEN real time PCR (Roche) using mRNA specific primer pairs and b-actin as a reference (Table I, section 4).

Flow Cytometry.

GFP and RFP expression in Jurkat 2D10 cells was quantified in live cells using a Guava EASYCYTE Mini flow cytometer (Guava Technologies). For HIV-1 reporter virus titer, HEK 293T cells were trypsinized 48 h after infections, washed and fixed in 4% paraformaldehyde for 10 minutes, then washed 3 times in PBS and analyzed for GFP FACS. CD4 expression in primary T cells was checked by direct labeling with CD4 V5 FITC antibody (BDBiosciences) followed by FACS.

Anexin Assay.

Jurkat cells were washed, counted and diluted to a density of 1×10⁵ cells/ml in PBS. For each sample, 100 μL of cells in suspension was mixed with 100 μl of room-temperature annexin V-PE staining reagent (Guava Nexin Reagent) and incubated for 20 minutes at room temperature in the dark. After incubation, samples were acquired using a Guava EasyCyte Mini flow cytometer.

Cell viability was assessed using propidium iodide staining. To 200 μl of live cells in suspension, PI solution was added to final concentration 10 μg/ml. Samples were incubated for 5 minutes at room temperature in the dark. After incubation, samples were acquired using a Guava EASYCYTE Mini flow cytometer.

Cell Cycle Analysis.

Cells were washed with 1x PBS and then resuspended in 250 μl of room temperature 1x PBS. This suspension was added drop-wise to Iml of −20° C. 88% ethanol, for a final concentration of 70% ethanol. Cells were fixed overnight at −20° C. then washed, incubated with 10 μg/ml of propidium iodide and RNase A solution, 100 g/ml, in 1x PBS for 30 minutes at 37° C. The samples were then cooled down at 4° C. and acquired using a Guava EASYCYTE Mini flow cytometer.

Western-Blot, Immunocytochemistry.

Whole cell lysates were prepared by incubation of Jurkat cells in TNN buffer (50 mM Tris pH 7.4, 150 mM NaCl, 1% Nonidet P-40, 5 mM EDTA pH 8, 1x protease inhibitor cocktail for mammalian cells (Sigma)) for 30 minutes on ice, then precleared by spinning at top speed for 10 minutes at 4° C. 50 μg of lysates were denatured in 1x Laemli buffer and separated by SDS-polyacrylamide gel electrophoresis in Tris-glycine buffer, followed by transfer onto nitrocellulose membrane (BioRad). The membrane was blocked in 5% milk/PBST for 1 h and then incubated with mouse anti-flag M2 monoclonal antibody (1:1000, Sigma) or mouse anti-α-tubulin monoclonal antibody (1:2000). After washing with PBST, the membranes were incubated with conjugated goat anti-mouse antibody (1:10,000) for 1 h at room temperature. The membranes were scanned and analyzed using an Odyssey Infrared Imaging System (LI-COR Biosciences). Cells were cultured in 4-well chamber slides and next day fixed with 4% paraformaldehyde/PBS for 10 min. After 3 times washing, cells were incubated in 0.1% Triton X-100, 2% BSA/PBS with mouse anti-flag M2 monoclonal antibody (1:1000, Sigma) at room temperature for 2 h. After washing 3 times, cells were incubated with goat anti-mouse FITC secondary antibody (1:200), and then incubated with Hoechst 33258 for 5 min. After 3 rinses with PBS, the cells were coverslipped with anti-fading aqueous mounting media (Biomeda) and analyzed under a Leica DMI6000B fluorescence microscope.

Statistical Analysis

The represented ±SD were from three experiments and were evaluated by student t test or ANOVA and Newman-Keals multiple comparison test. In general, a p value <0.05 or 0.01 was considered as statisically significant.

Table 1 shows the sequences of DNA oligonucleotides used in this study.

TABLE 1

| Primer | Sequence |
|---|---|
| 1. PCRs | |
| LTR −453/S | 5′-TGGAAGGGCTAATTCACTCCCAAC-3′ (SEQ ID NO: 7) |
| LTR −374/S | 5′-TTAGCAGAACTACACACCAGGGCC-3′ (SEQ ID NO: 8) |
| LTR +43/AS | 5′-CCGAGAGCTCCCAGGCTCAGATCT-3′ (SEQ ID NO: 8) |
| LTR −417/S | 5′-GATCTGTGGATCTACCACACACA-3′ (SEQ ID NO: 10) |
| LTR −19/AS | 5′-GCTGCTTATATGTAGCATCTGAG-3′ (SEQ ID NO: 11) |
| RRE/S | 5′-CGCCAAGCTTGAATAGGAGCTTTGTTCC-3′ (SEQ ID NO: 12) |
| RRE/AS | 5′-CTAGGATCCAGGAGCTGTTGATCCTTTAGG-3′ (SEQ ID NO: 13) |
| LTR-A-OT-1/S | 5′-GTGGACTTTGGATGGTGAGATAG-3′ (SEQ ID NO: 14) |
| LTR-A-OT-1/AS | 5′-GCCTGGCAAGAGTGAACTGAGTC-3′ (SEQ ID NO: 15) |
| LTR-A-OT-2/S | 5′-AAGATAATGAGTTGTGGCAGAGC-3′ (SEQ ID NO: 16) |
| LTR-A-OT-2/AS | 5′-TCTACCTGGTAATCCAGCATCTGG-3′ (SEQ ID NO: 17) |
| LTR-A-OT-3/S | 5′-ATAGGAGGAAGGCACCAAGAGGG-3′ (SEQ ID NO: 18) |
| LTR-A-OT-3/AS | 5′-AATGATGCTTTGGTCCTACTCCT-3′ (SEQ ID NO: 19) |
| LTR-A-OT-4/S | 5′-TGCTCTTGCTACTCTGGCATGTAC3′ (SEQ ID NO: 20) |
| LTR-A-OT-4/AS | 5′-AATCTACCTCTGAGAGCTGCAGG-3′ (SEQ ID NO: 21) |
| LTR-A-OT-5/S | 5′-TCAGACACAGCTGAAGCAGAGGC-3′ (SEQ ID NO: 22) |
| LTR-A-OT-5/AS | 5′-ATGCCAGTGTCAGTAGATGTCAG-3′ (SEQ ID NO: 23) |
| LTR-A-OT-6/S | 5′-TCAAGATCAGCCAGAGTGCACATG-3′ (SEQ ID NO: 24) |
| LTR-A-OT-6/AS | 5′-TGCTCTTCCGAGCCTCTCTGGAG-3′ (SEQ ID NO: 25) |
| b-actin S | 5′-CTACAATGAGCTGCGTGTGGC-3′ (SEQ ID NO: 26) |
| b-actin AS | 5′-CAGGTCCAGACGCAGGATGGC-3′ (SEQ ID NO: 27) |
| 2. Long range PCR | |
| D10-Chr1-5′ArM/F(6-29) | 5′-GAGCACAGGACTCATTCAACAGT-3′ (SEQ ID NO: 28) |
| D10-Chr1-3′Arm/R(276- | 5′-TTTGTATGTCAACAGACAGTATCCAG-3′ (SEQ ID NO: 29) |
| 250) | |
| D10-Ch16 MSRB1-S | 5′-TGTGCATACTTCGAGCGGCT-3′ (SEQ ID NO: 30) |
| D10-Ch16 MSRB1-AS | 5′-GGAAAGGCGGGAGCTGATGA-3′ (SEQ ID NO: 31) |
| 3. gRNA RT and PCR | |
| pX260-crRNA-3′/R | 5′-TGGGACCATTCAAAACAGCAT-3′ (SEQ ID NO: 32) |
| 4. Neighboring genes | |
| qPCR | |
| RSBN1/F | 5′-GTAAGGCCAGGAGAACAGATG-3′ (SEQ ID NO: 33) |
| RSBN1/R | 5′-TCAAAGAGAACTTCGCGGG-3′ (SEQ ID NO: 34) |
| PHTF1/F | 5′-CCCAAGTTGTGTCCATCCTATC-3′ (SEQ ID NO: 35) |
| PHTF1/R | 5′-AGACACCCCATTACCCAAAC-3′ (SEQ ID NO: 36) |
| MAGI3/F | 5′-GACACCGCAGTAATTTCAGTTG-3′ (SEQ ID NO: 37) |
| MAGI3/R | 5′-AGCAAGACGAAGGATGAACAG-3′ (SEQ ID NO: 38) |
| PTPN22/F | 5′-TTTGCCCTATGATTATAGCCGG-3′ (SEQ ID NO: 39) |
| PTPN22/R | 5′-GTTGTAGATAAAGGACCCTGGG-3′ (SEQ ID NO: 40) |
| AP4B1-AS1/F | 5′-AGAAGGAAAAGGAGCAGACAC-3′ (SEQ ID NO: 41) |
| AP4B1-AS1/R | 5′-AGAAAGTGGAGGTGCTGTG-3′ (SEQ ID NO: 42) |
| HS3ST6/F | 5′-CTTCTACTTCAACGCCACCA-3′ (SEQ ID NO: 43) |
| HS3ST6/R | 5′-AAGGGCCGGTAGAACTCC-3′ (SEQ ID NO: 44) |
| RPL3L/F | 5′-AACAATGCATCCACCAGCTA-3′ (SEQ ID NO: 45) |
| RPL3L/R | 5′-GTAATGACCCGCTTCTTGGT-3′ (SEQ ID NO: 46) |
| MSRB1/F | 5′-GAAGCTTAGGCCCACATCTC-3′ (SEQ ID NO: 47) |
| MSRB1/R | 5′-CTGGAAGGGTTTGACCAGAG-3′ (SEQ ID NO: 48) |
| NDUFB10/F | 5′-GCATGTATGAAGCCGAAATG-3′ (SEQ ID NO: 49) |
| NDUFB10/R | 5′-TGAACTGCTCCACTTCCTTG-3′ (SEQ ID NO: 50) |
| RPS2/F | 5′-GCCTCTCTCAAGGATGAGGT-3′ (SEQ ID NO: 51) |
| RPS2/R | 5′-CAACAAATGCCTTGAACCTG-3′ (SEQ ID NO: 52) |
| b-actin S | 5′-CTACAATGAGCTGCGTGTGGC-3′ (SEQ ID NO: 53) |
| b-actin AS | 5′-CAGGTCCAGACGCAGGATGGC-3′ (SEQ ID NO: 54) |
| 5. Target A and B | |
| oligos and cloning | |
| LTR-A S/F/5′ | 5′-CACCGATCAGATATCCACTGACCTT-3′ (SEQ ID NO: 55) |
| LTR-A S/R/3′ | 5′-AAACAAGGTCAGTGGATATCTGATC-3′ (SEQ ID NO: 56) |
| LTR-B AS/F/5′ | 5′-CACCGCAGCAGTTCTTGAAGTACTC-3′ (SEQ ID NO: 57) |
| LTR-B AS/R/3′ | 5′-AAACGAGTACTTCAAGAACTGCTGC-3′ (SEQ ID NO: 58) |
| T560 | 5′-TATGGGCCCACGCGTGAGGGCCTATTTCCCATGATTCC-3′ (SEQ |
| | ID NO: 59) |
| T561 | 5′-TGTGGATCCTCGAGGCGGGCCATTTACCGTAAGTTATG-3′ (SEQ |
| | ID NO: 60) |
| 6. Taqman qPCR | |
| HIV-Gag-RTfw | 5′-CATGTTTTCAGCATTATCAGAAGGA-3′ (SEQ ID NO: 61) |
| HIV-Gag-RTrev | 5′-TGCTTGATGTCCCCCCACT-3′ (SEQ ID NO: 62) |
| HIV-RTprobe | 5′-/56-FAM/-CCACCCCACAAGATTTAAACACC-BHQ-3′ (SEQ ID NO: 63) |
| b-globinRTfw | 5′-CCCTTGGACCCAGAGGTTCT-3′ (SEQ ID NO: 64) |
| b-globinRTrev | 5′-CGAGCACTTTCTTGCCATGA-3′ (SEQ ID NO: 65) |
| b-globinRT probe | 5′-FAM-GCGAGCATCTGTCCACTCCTGATGCTGTTATGGGCGCTCGC- |
| | TAMRA-3′ (SEQ ID NO: 66) |

### Example 2: A CRISPR/Cas9 System for Inhibiting the Reactivation of Latent HIV-1 in Human T Lymphocytic Cells

Cas9/gRNA Inhibits HIV-1 Reactivation of Latent HIV-1 in Human T-Cells.

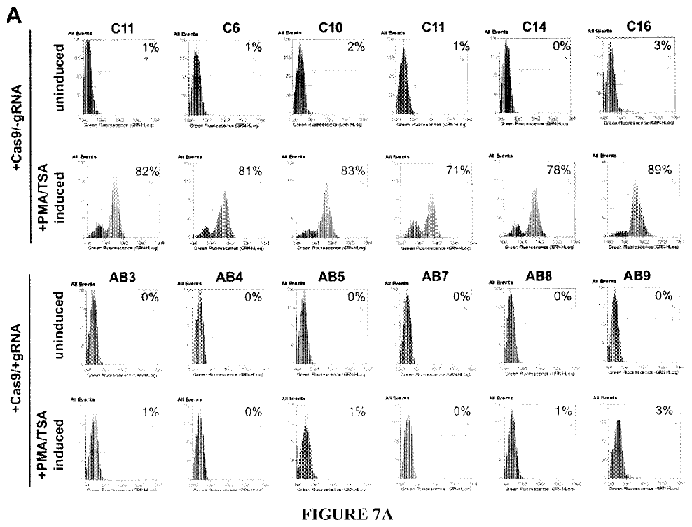

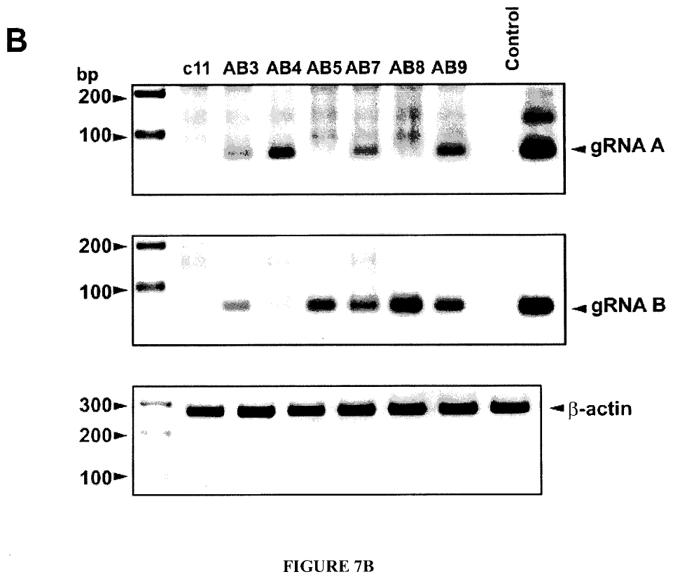

Initial experiments were performed with the aim of determining whether the CRISPR/Cas9 system according to the present invention can eliminate the HIV-1 genome in a human T-lymphocytic cell line, 2D10. These cells harbor integrated copies of a single round HIV-1P_{NL4-3} whose genome lacks sequences encoding the majority of the Gag-Pol polyprotein, but encompasses the full-length 5′ and 3′ LTRs, and includes a gene encoding the marker protein green fluorescent protein (GFP) replacing Nef protein in the latent state (FIG. 1A). Thus, 2D10 is a suitable cell line to first establish proof-of-principle of HIV-1 eradication because of the uniform nature of the integrated provirus. Treatment of clonal 2D10 cells stably expressing Cas9, but not gRNAs, with proinflammatory agents such as phorbol myristate acetate (PMA) and/or the HDAC inhibitor trichostatin A (TSA) profoundly stimulates HIV-1 promoter activity, leading to production of the viral proteins and GFP in over 90% of treated cells (FIG. 1B, left panels), providing a convenient cell culture model for studying viral latency and reactivation. Co-expression of Cas9 along with gRNAs A and B, designed respectively to target the highly conserved sequence among all viral isolates spanning the LTR U3 region at nt-287/-254 (gRNA A) and nt-146/-113 (gRNA B) (FIG. 1A) completely eliminated PMA/TSA-induced GFP production, indicating inhibition of HIV-1 gene expression in the pre-selected mixed clonal population of T-cells expressing both Cas9 and gRNA expression plasmids (FIG. 1B, right panels). Expression of gRNAs and Cas9 was verified by RT-PCR and Western blot, respectively (FIGS. 1C, 1D). HIV-1 expression was completely eliminated from the cells expressing both Cas9 and gRNA expression plasmids, shown by flow cytometry detection of GFP production by randomly-selected Cas9-positive clonal cells with or without gRNA expression (FIG. 7A). Also, it was found that GFP production was effectively blocked in many clones that expressed only a single gRNA (A or B), to levels similar to those elicited by co-expression of both A and B (FIG. 7B; also see FIG. 7A), evidencing that expression of either gRNA in single configuration can initiate cleavage at both LTRs to achieve eradication of proviral DNA.

Integration Sites of HIV-1 Proviral DNA in Human T-Cells and Excision of Viral DNAs from Host Cell Chromosomes.

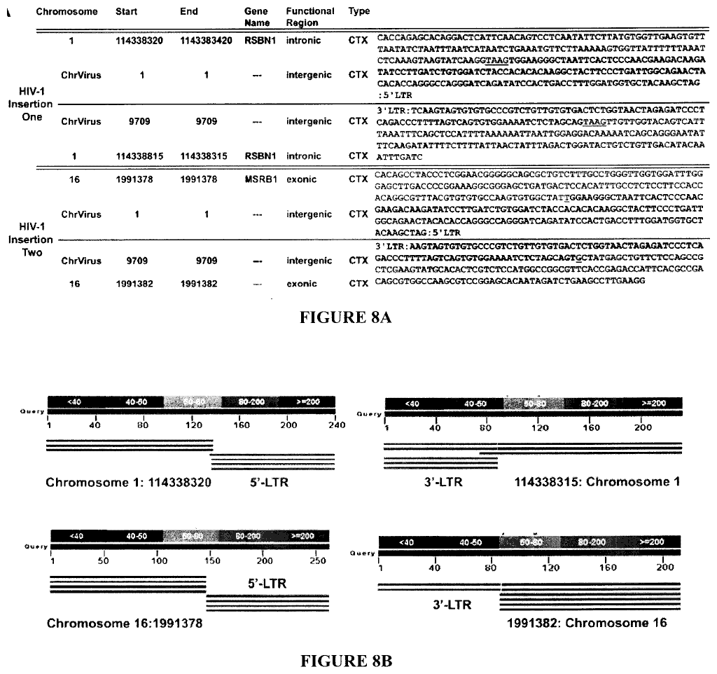

The site(s) of HIV-1 proviral DNA integration were verified by whole-genome sequencing (WGS) of 2D10 cells. CREST (“clipping reveals structure”) calling (Wang, Mulligham, et al., Nat Methods 8, 652-654 (2011)) of structural variation (SV) was employed to investigate the breakpoints caused by proviral DNA integration in the host genome, and used the hgl9 genome and the HIV-1 genome, KM390026.1 as reference genomes for reading the DNA sequences. Four inter-chromosomal translocations were identified, designated by CTX (FIGS. 8A, 8B), that are related to HIV-1 DNA. Breakpoints between the HIV-1 5′ LTR and P163.3:1991382 and the HIV-1 3′ LTR P613.3:1991378 were detected, mapping to exon 2 of the methionine sulfoxide reductase B1 MSRB1 gene (NM_01332), and corresponding to a previously mapped location for the provirus in the 2D10 cells (Pearson, R. et al. J Virol 76, 11091-11093 (2002); Jadlowsky, J. K. et al. Mol Cell Biol 34, 1911-1928 (2014)). In addition, two CTXs were mapped to chromosome 1 with the breakpoint between P13.2:114338315 and the HIV-1 5′ LTR, and other breakpoints between HIV-1 3′ LTR and P13.2:114338320. Also, it was noted that four nucleotides, TAAG, were deleted between the two breakpoints in chromosome 1P13.2. The HIV-1 provirus in chromosome 1, which was previously undetected by linker-addition mapping, was integrated in the second intron (114339984-114320431) of the round spermatid basic protein 1 (RSBN1) gene (NM_018364). A schematic presentation of identified consensus sequences for sites of HIV-1 DNA integration in chromosomes 1 and 16 are shown in FIGS. 8A, 8B.

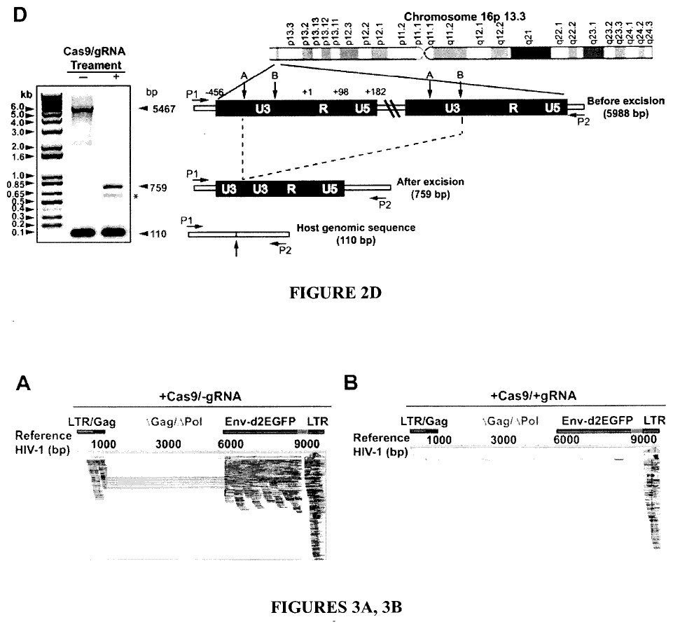

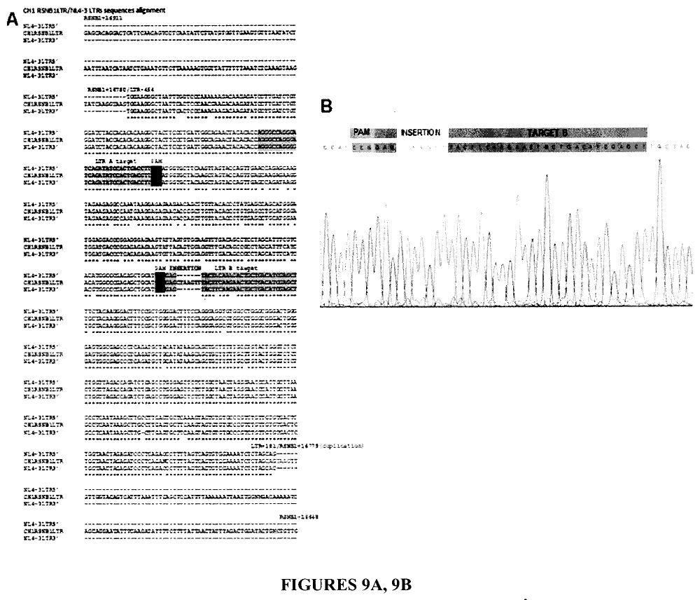

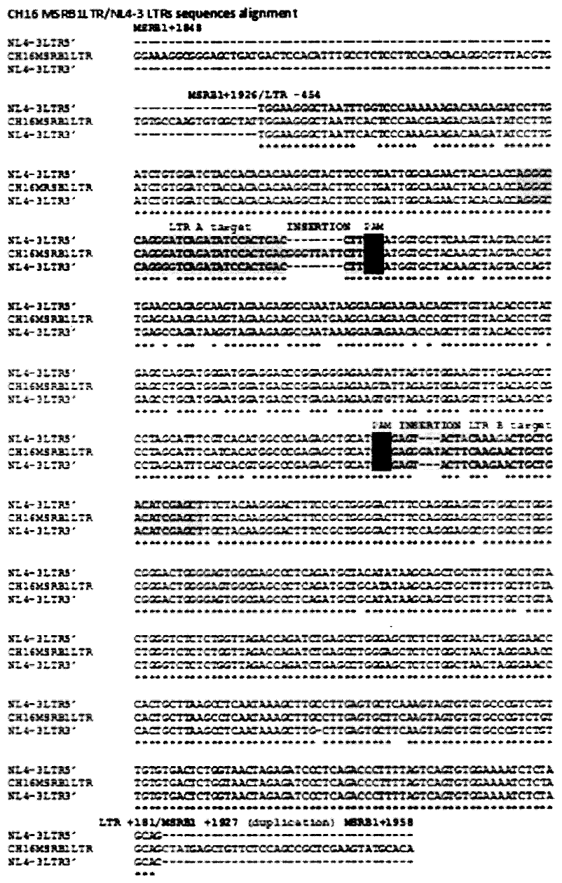

Short-range amplification assay of LTR DNA revealed an expected 497-bp DNA fragment in control cells and a second DNA fragment of similar size (504 bp) after treatment with Cas9/gRNAs A and B (FIG. 2A). Results of direct DNA sequencing of the PCR amplicon provided evidence that the observed 504-bp DNA fragment in Cas9/gRNA-treated cells was created by joining of the residual 5′ LTR to the remaining 3′ LTR after cleavage by Cas9/gRNA B (FIG. 2B). An Indel mutation with a seven-nucleotide insertion was also detected in the junction of the 5′ and 3′ fusion site of the clonal cells (FIG. 2B). The 257-bp PCR amplicon corresponding to the Rev response element (RRE), which is positioned in the center of the viral genome, was absent, verifying that Cas9/gRNAB removed the DNA sequences spanning between the two terminal repeats (FIG. 2A). Long-range PCR analysis of 2D10 control cells expressing Cas9 but not gRNAs, using a pair of primers derived from the second intron of RSBN1, verified the presence of a 6130-bp DNA fragment corresponding to the integrated HIV-1 genome plus its chromosome 1-derived flanking DNA sequence (FIG. 2C). The 264-nucleotide DNA fragment that represents host cell DNA sequence from the other copy of chromosome 1 was also present (shown at the bottom of the gel). In cells treated with Cas9/gRNAs A and B, a DNA fragment of 6130 nucleotides corresponding to the integrated HIV-1 genome was completely absent. Instead, PCR amplification produced a smaller DNA fragment of 909 nucleotides. Sequencing of the amplicon verified excision of the integrated viral DNA, spanning between the B domain of the 5′ LTR and the B domain of the 3′ LTR (FIGS. 9A, 9B). Again, a 264-nucleotide DNA fragment amplified from the host genome from the other chromosome, was detected (FIG. 2C).

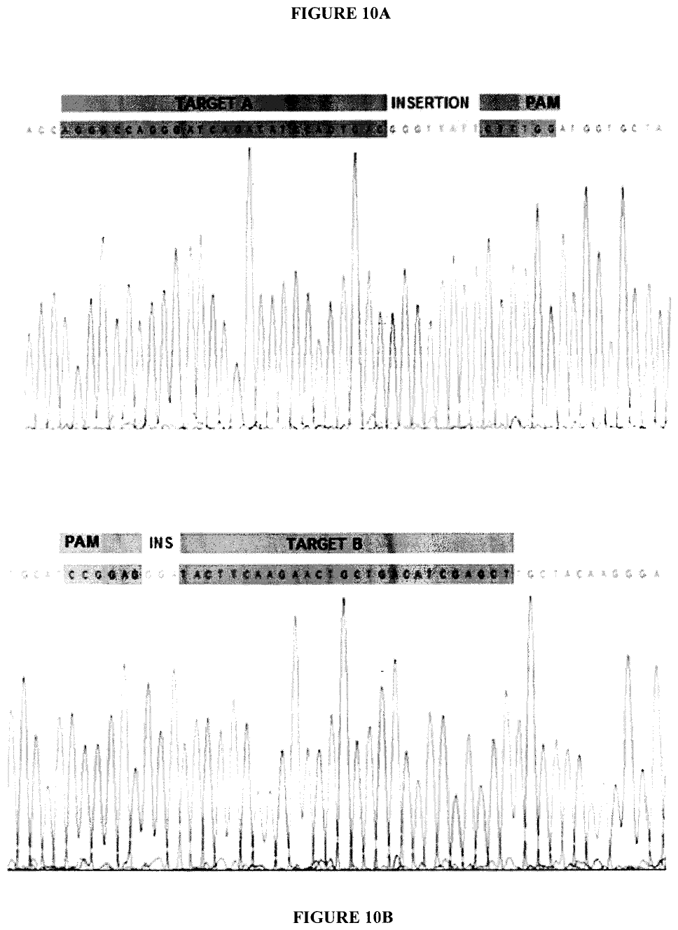

Chromosome 16 was examined for presence of HIV-1 proviral DNA using long-range PCR using a primer pair corresponding to the second exon of MSRB1 gene and compared its status in Cas9/gRNA A/B-treated cells. The results showed that the expected 5467-bp DNA fragment of the HIV-1 genome and its flanking host DNA in chromosome 16 was absent. Instead a smaller 759-bp DNA fragment was detected, that reflected joining of the residual U3 region of the 5′ LTR after cleavage by gRNA A to the remaining U3 region of the 3′ LTR upon cleavage by gRNA B (FIG. 2D). Direct sequencing of the 759-bp DNA fragment identified the sites of viral DNA excision (FIGS. 10A, 10B). A smaller, 110-bp DNA fragment found resulted from amplification of host DNA from the other copy of chromosome 16. These observations provide strong evidence that the gene editing molecules used effectively eliminate multiple copies of the integrated proviral DNA of the HIV-1-genome, which are scattered among various chromosomes.

Elimination from Host Cells of HIV-1 DNA Sequence Spanning Between 5′ and 3′ LTRs, and Positions of the Breakpoints.

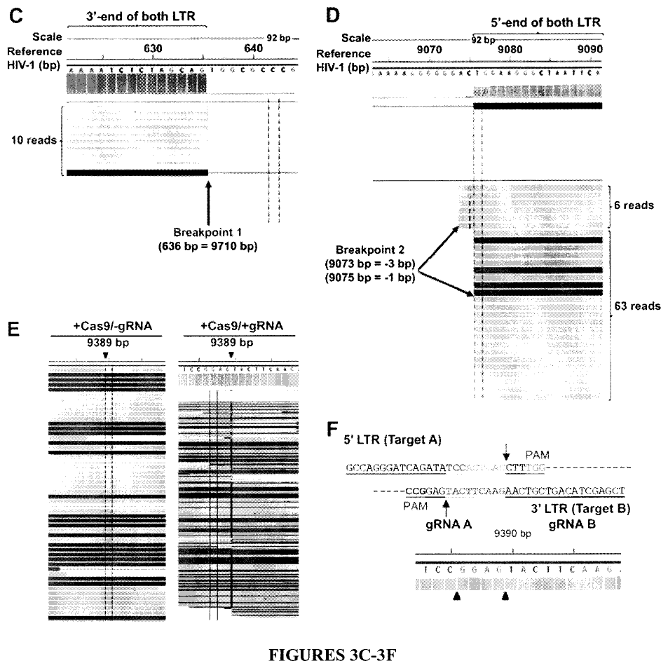

To further validate the efficiency of the Cas9/gRNA treatment-based gene editing strategy in eliminating HIV-1 proviral DNA from latently-infected T-cells, the occurrence of insertion/deletion (InDel) and single nucleotide polymorphisms (SNP) in the HIV-1 genomes of control and HIV-1-eradicated cells, was analyzed using GATK calling (Depristo, et al., Nat Genetics 43, 491-498 (2011)) against reference HIV-1 DNA (GenBank accession #KM3900261). Consistent with the results shown in FIGS. 2A-2D, the reads from the whole-genome sequencing mapped to the 5′- and 3′-LTRs and to the proviral genome in the control 2D10 cells, supporting the precision and reliability of the deep coverage of the HIV-1 DNA by genome sequencing (FIG. 3A). In Cas9/gRNA-treated 2D10 cells, the integrated genomics view (IGV) revealed complete removal of a large DNA fragment corresponding to the HIV-1 proviral DNA with reads that map to the 3′ LTR (FIG. 3B). Reads mapping to the entire proviral genome between the two LTRs were completely absent, evidencing that both copies of the integrated HIV-1 genome in host cells were fully eliminated, and that Cas9/gRNA expression in a single clonal cell can attain 100% gene editing/elimination, perhaps attributable to repeated genome editing by stably-expressed Cas9/gRNA. Further, these results evidence that, after cleavage of viral 5′ and 3′ LTRs and excision of the viral genome, viral DNA is likely degraded, such that no transposition or re-integration occurs into the host genome.

To determine the repair events after Cas9/gRNA A/B-induced cleavage of both LTRs, BWA calling (Wang, et al., Nat Methods 8, 652-654 (2011)) of the structural variant (SV) in the DNA from cells with HIV-1 excision, was used and which identified the breakpoints of large insertions and/or deletions. The results verified that no excised HIV-1 DNA from one chromosome was inserted in the host genome and/or in the integrated copy of proviral DNA on the other chromosome further ruling out the notion of re-integration of the excised viral DNA into host cell genome. However, three breakpoints were identified which were caused by deletion of the DNA fragments corresponding to sites of viral DNA integration into the host genome. One left breakpoint positioned at the end of the 5′ LTR at nucleotide 636 (=HIV: 9710) as supported by 10 reads. One right breakpoint exhibited two patterns, one at HIV: 9073 (=HIV:-3) supported by 6 reads with 2 C→G and 4 C→T conversions; and HIV: 9075 (=HIV:-1) supported by 63 reads (FIG. 3C,D). Of note, these two breakpoints can actually reflect the presence of the entire 634 nucleotides of the LTR after the excision of full proviral DNA by Cas9/gRNAs A or B at the 5′ and 3′ LTRs followed by precise rejoining of the DNA at the cleavage site. A third breakpoint is located in the middle of the 3′ LTR at nucleotide 9389 (=HIV: 313) with C insertion supported by 87/161 reads and CTAAGTT insertion supported by 69/161 reads (FIG. 3E). This breakpoint represents the joining of DNA after cleavage at sites A and B of the 5′ and 3′ LTRs (FIG. 3F).

Effect of Excision of HIV-1 Proviral DNA on the Neighboring Gene Expression and Off-Target Effects.

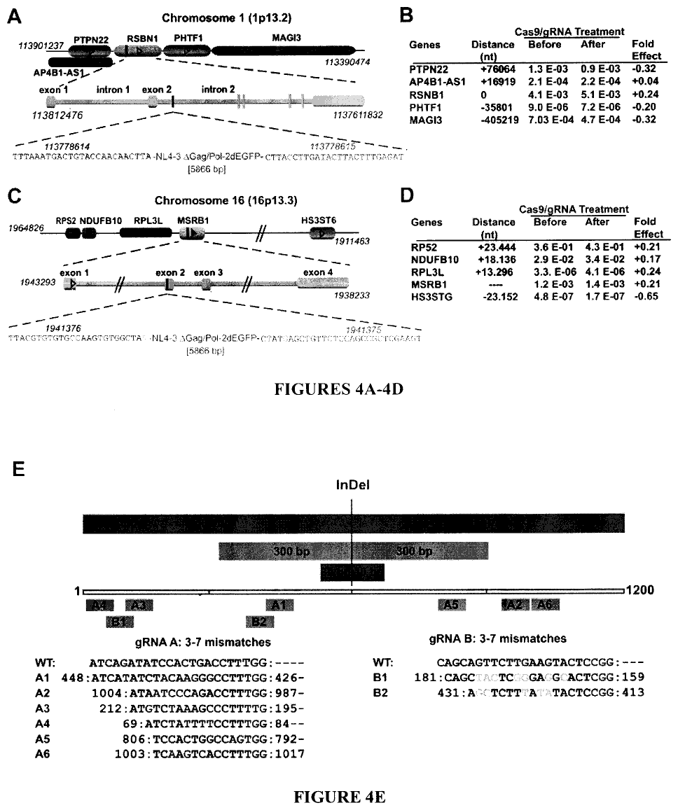

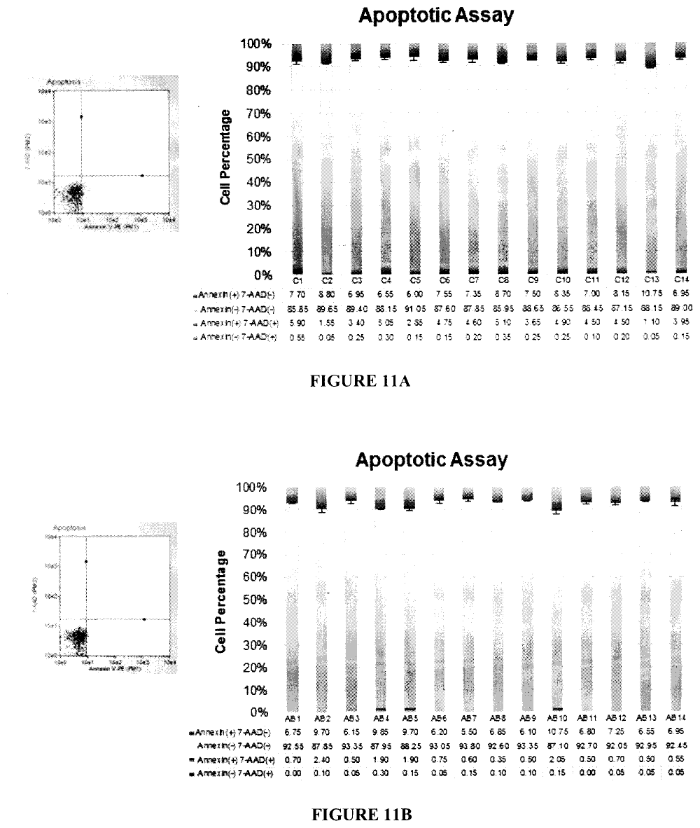

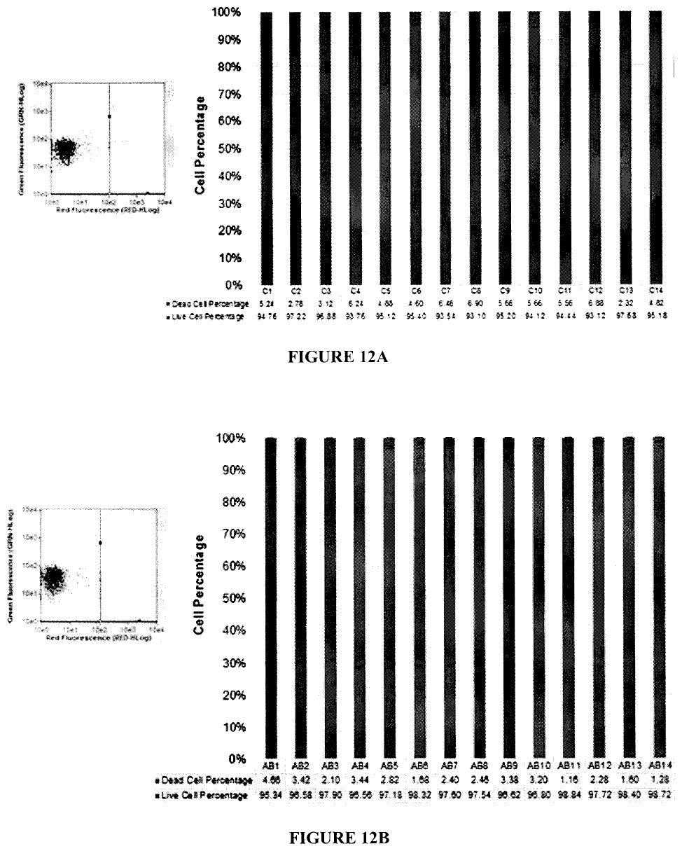

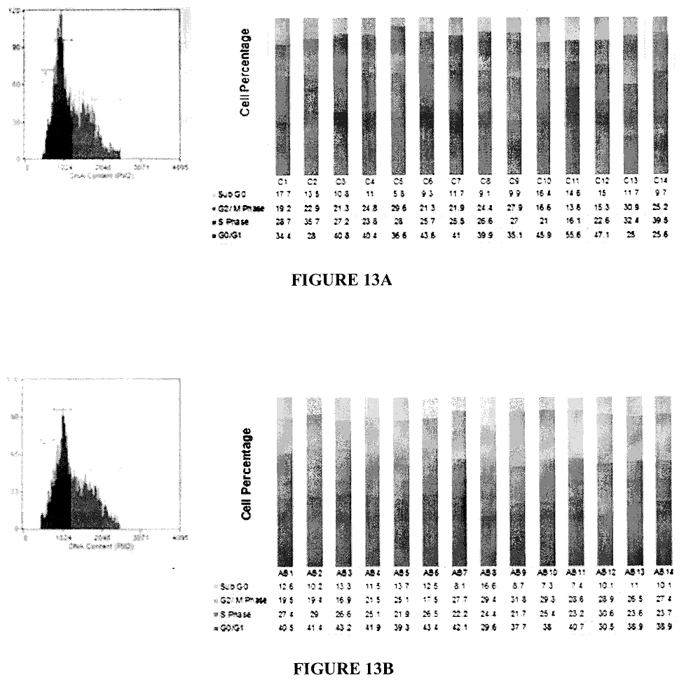

The impact of CRISPR/Cas9-mediated excision of HIV-1 proviral DNA from the RSBN1 gene was investigated. The level of RNA production from RSBN1 and several of the other cellular genes positioned in close proximity of the proviral insertion site was determined, as shown in FIG. 4A. Results from RT-PCR of five controls and five HIV-1 eradicated single cell clones indicated no significant effect on the level of expression of RSBN1, although smaller variations of less than 0.4-fold were detected in the levels of neighboring RNA (FIG. 4B), which may not be attributed to the gene editing strategy and may not impact the overall expression of their proteins. Similarly, elimination of the HIV-1 genome from chromosome 16 showed no significant impact on the expression of the site of integration, i.e. MSRB1 gene and its surrounding gene (FIGS. 4C, 4D). The effect of Cas9/gRNAs A and B was investigated based on several parameters related to the health of the cells, including cell viability, cell cycle progression and apoptosis using several clonal cells after the eradication of HIV-1 by Cas9/gRNAs A and B. No persistent and significant deleterious effects were found on the host cells vital signs after elimination of the HIV-1 proviral DNA by the Cas9/gRNAs A and B (FIGS. 11A, 11B, 12A, 12B, 13A, 13B).

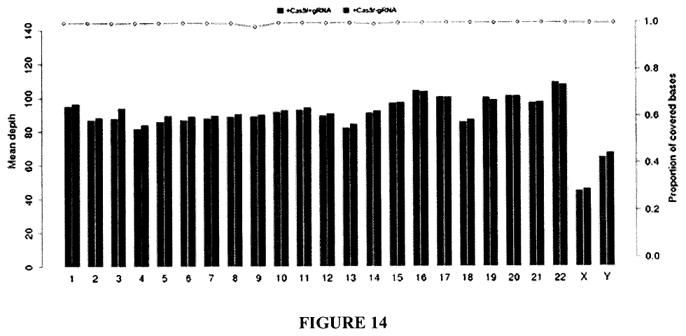

To expand the scope of analysis of potential off-targets, the InDel results from whole-genome sequencing of the 2D10 cells after treatment with the Cas9/gRNAs A/B system that elicited complete eradication of the proviral HIV-1 DNA, were compared. To improve InDel-calling confidence, the whole-genome sequencing at 100x coverage was sought for, but statistical analyses revealed that the actual achieved total coverage was 109.3x for control cells and 112.7x for HIV-1 eradicated cells (Table 2). Coverage levels varied for each chromosome, ranging >96x for chromosome 1 and >110x for chromosome 16 (FIG. 14). Using human (hgl9) genome as a reference sequence, 1,361,311 InDels (<50 bp insertions/deletions) were identified in Control (+Cas9/−gRNA) and 1,358,399 in HIV-1-eradicated cells (Table 3), and 3,973,098 single nucleotide polymorphisms (SNP) in Control and 3,961,395 in HIV-1-eradicated cells (Table 4). Comparative bioinformatics analysis between the control and the HIV-1-eradicated cells identified 32,399 somatic InDels (small insertion/deletion called by Strelka), 46,614 somatic SNVs (single-nucleotide variations, called by MuTect) and 52 SVs (structural variations including large InDels called by CREST) between the latter two groups, that were distributed in different genomic regions (Table 5).

After discarding the small InDels found in the public database dbSNP, 30,156 InDels and 43,858 SNVs were identified in HIV-1-eradicated cells. Filtering out heterozygous mutations, reduced this number to 989 InDels. To determine if these filtered InDels are de novo mutations caused by the Cas9/gRNA A/B editing system, ±30 bp, +300 bp or ±600 bp sequences flanking each filtered InDel were extracted and Blastn (e-value cutoff: 1000) was used to compare them vs. the potential gRNA off-target host genome sites predicted by sequence similarity at 0-7 mismatches, and vs. HIV-1 on-target sequences. Without any mismatches to targets of gRNAs A and B, no off-target site was found around the extracted 60, 600 and 1200 bp sequences of the filtered InDels. Within the extracted 60-bp sequences, no off-target site was found even with 7 mismatches at alignment lengths >12 nucleotides from PAM NRG (which must be 100% matched). Within the extracted 600-bp sequences, no off-target site with 3 mismatches was found for targets of gRNA A or B. With 4-7 mismatches, only one potential off-target site was found with 6 mismatches at an alignment length of 20 bp from PAM and another with 3 mismatches at 12 bp alignment length from PAM for Target A, and one additional potential off-target site with 4 mismatches at 16 bp length from PAM for Target B. Within the extracted 1200 bp sequences for 3 mismatches, no off-target sites were found for Target A but one potential off-target with 2 mismatches at 13 bp from PAM for Target B. With criteria of 3-7 mismatches against the 1200-bp sequences, only six potential off-target sites for Target A and two potential off-target sites for Target B were found (FIG. 4E). Together, these data provide strong evidence that none of the indels detected in the cells with the excised HIV-1 genome lie within 60 bp of Targets A or B of any potential off-target sites, as predicted by search criteria allowing up to 7 mismatches. By expanding the searching sequences to 600 or 1200 bp, relatively rare off-target sites were identified, including various numbers of mismatches and aligned length. With perfect match to the last 12 bp seed sequence plus PAM NRG, none of the indels fell within the search area of 60-1200 DNA sequences. The overall interpretation of these data verifies the preceding Surveyor assay results in these cells, as well as in the other cell types (W Hu, et al. Proc Natl Acad Sci USA 111, 11461-11466 (2014)), and establishes by very stringent analysis that no off-target effects upon the host T cell genome is elicited by the Cas9/gRNAs HIV-1 DNA-excising system.

Infectivity of the HIV-1 Eradicated Cells by HIV-1.

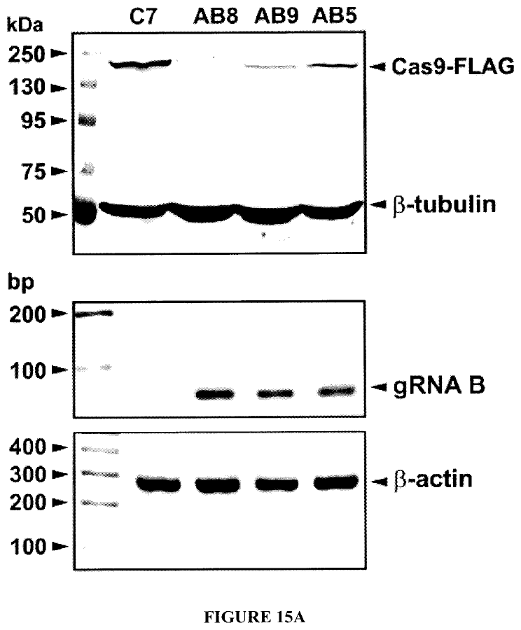

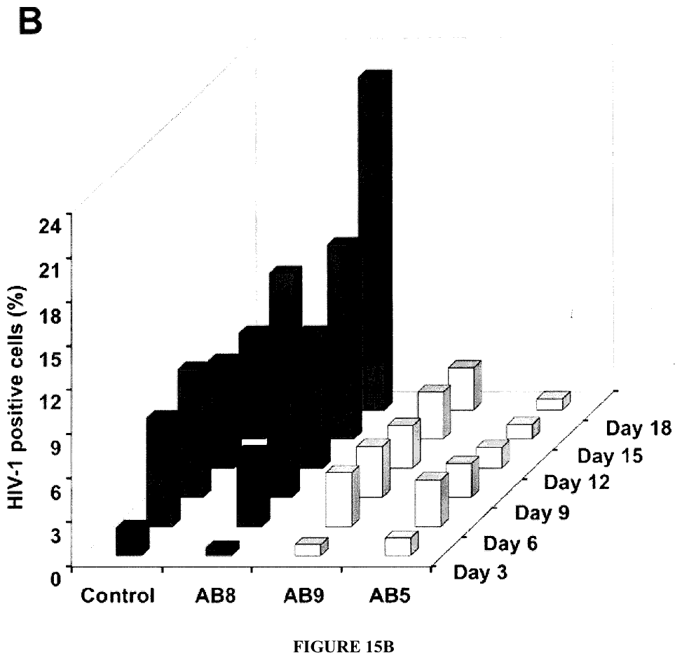

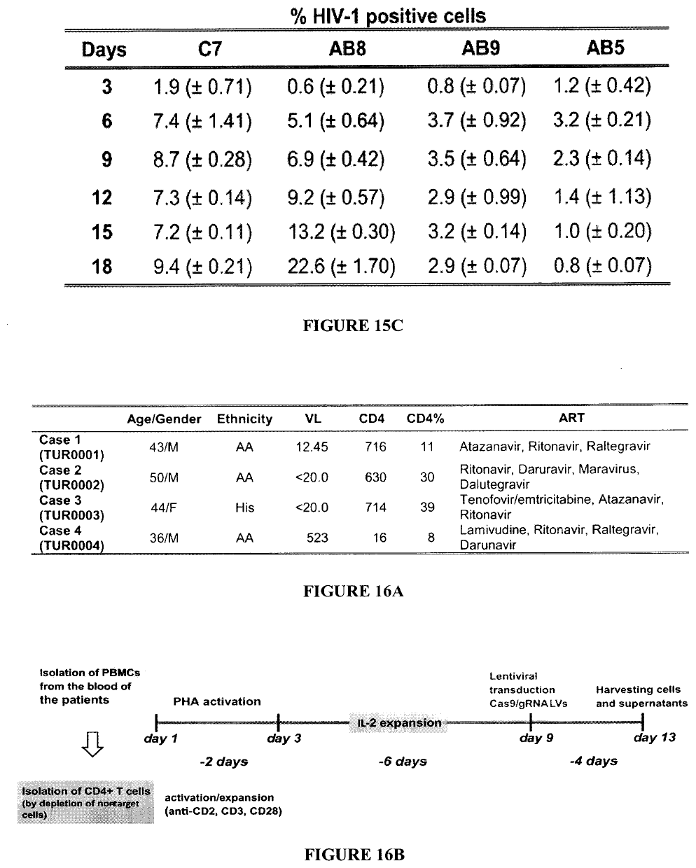

Several T-cell clones were selected whose proviral DNA was eliminated by Cas9/gRNAs and maintained at various levels, expression of Cas9 as well as the gRNAs to assess the extent of new infection by HIV-1. As seen in FIG. 15A, clone C7 expresses Cas9 but not gRNA B, whereas clone AB8 shows no detectable level of Cas9, yet contains gRNA B. Two additional clones AB9 and AB5 with an equal amount of gRNA B and different levels of Cas9 expression were selected for a re-infection study. Infection of these cells by HIV-1_{NL4}-g_{fp} followed by longitudinal evaluation of viral replication by flow cytometry showed that cells expressing either Cas9 or gRNA B alone were infectable by HIV-1, and supported viral replication throughout the course of these studies (day 18 post-infection) (FIG. 15B). In contrast, cells expressing both Cas9 and gRNA B were resistant to infection by HIV-1 and failed to support viral replication. AB5, which expressed a higher level of Cas9, appeared to be more resistant to viral replication than AB9, which showed reduced Cas9 expression (FIGS. 15A, 15B). FIG. 15C summarizes the quantitative values of the results shown in Panel B. The results demonstrate that the intracellular presence of both Cas9 and the LTR-directed gRNAs can effectively protect culture of human T-cells against new infection by HIV-1.

Lentivirus Mediated Delivery of Cas9/gRNA Suppresses HIV-1 Infection of Cd4⁺ T-Cells.

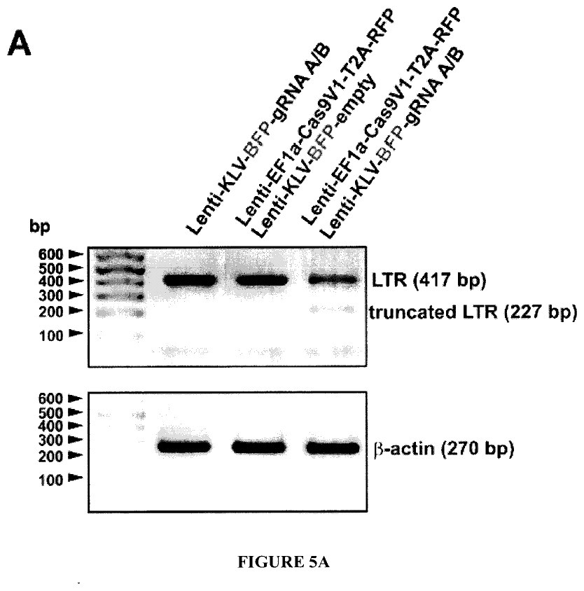

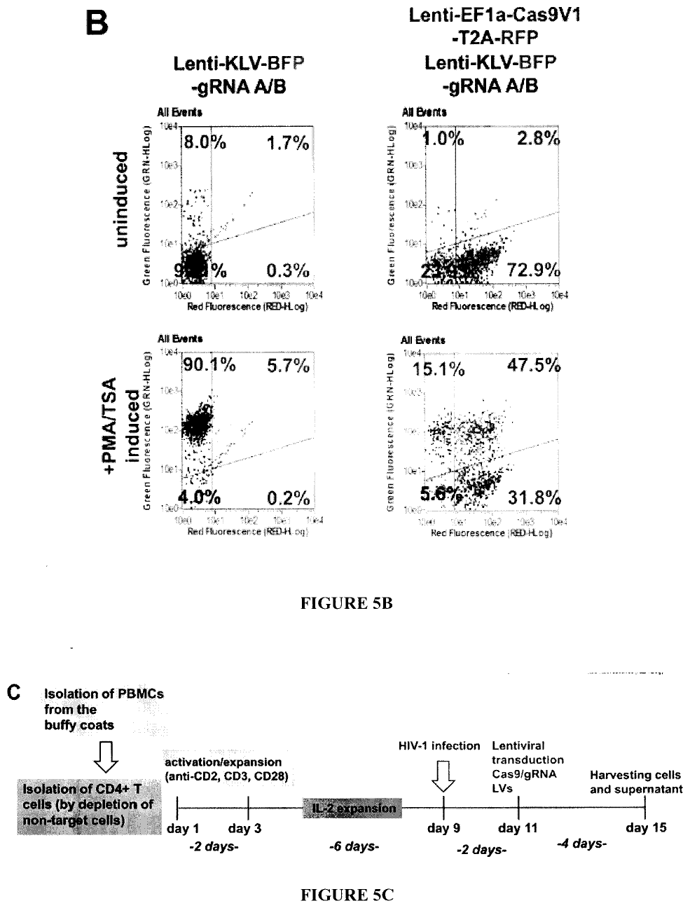

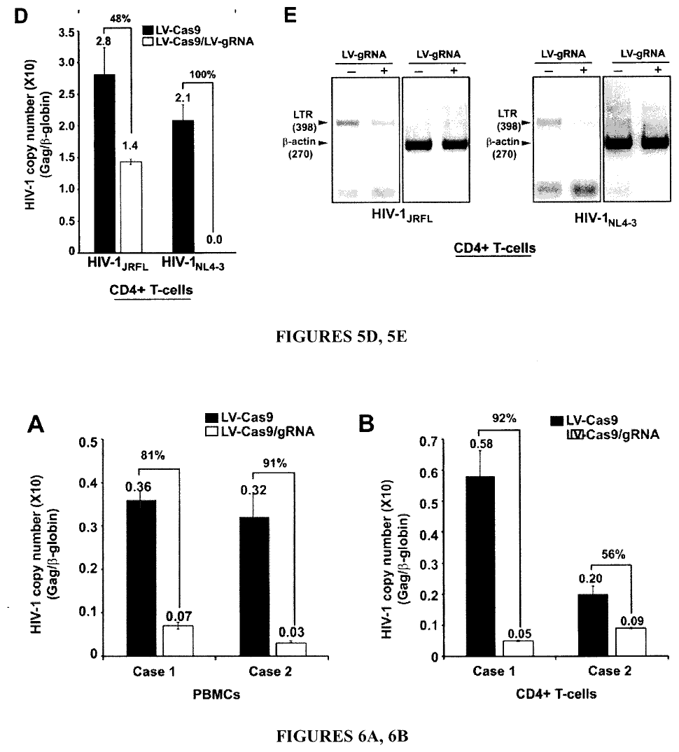

The ability of Cas9/gRNAs to suppress HIV-1 infection of CD4⁺ T-cells prepared from healthy individuals was tested. A lentivirus vector was chosen for delivering Cas9 and gRNA expression DNAs because of its high transduction efficiency and low toxicity. Results of the LV transduction showed efficient cleavage of the HIV-1 LTR DNA by the LVs expressing both Cas9 and gRNAs, but not in control cells transduced with LV expressing only Cas9 (FIG. 5A). Of note, the gRNAs do not cleave the LVs LTR, which lacks the U3 modulatory region, thus they have no effect on the expression of the LV genome. Accordingly, flow cytometry analysis revealed functional inactivation of the integrated HIV-1 genome in latently-infected T-cells upon transduction with LV-Cas9/gRNA (FIG. 5B). Again, no evidence of cell death was found that may be associated with Cas9/gRNAs in the primary cells, corroborating the observations shown in FIGS. 11A, 11B. Once the efficacy of the gene delivery of editing molecule by LV was verified in the T-cell line, primary cultures of CD4⁺ T-cells were infected with HIV-1_{JRFL} or HIV-1_{PNL4-3}, then transduced them with either control LV Cas9 or LV Cas9 plus LV gRNA (FIG. 5C). Compared to controls, a substantial decrease in HIV-1 copy number was seen in the CD4⁺ T-cells treated with LV Cas9/gRNA (FIG. 5D). Amplification of viral DNA revealed the expected 398-bp amplification in the control cells and a similar-sized DNA fragment with lesser intensity in cells transduced with LV Cas9/gRNA in CD4⁺ T-cells (FIG. 5E).

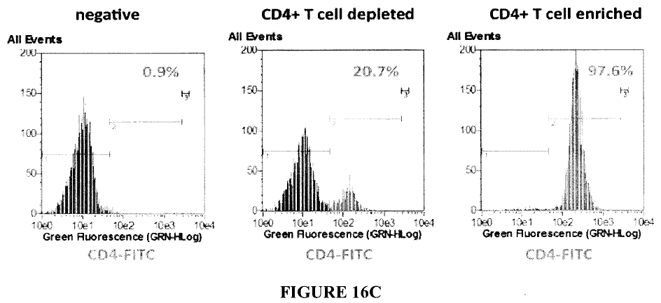

The HIV-1 genome editing ability of lentivirus delivered Cas9/gRNA was assessed in PBMC's and CD4⁺ T-cells, containing the HIV-1 genome, obtained from HIV-1⁺ patients during routine visits to the Temple University Hospital AIDS clinic. For this proof-of-concept study, initially it was initially sought to prepare PBMCs and CD4⁺ T-cells from four patients (TUR0001 to TUR0004; Cases 1-4) who were undergoing antiretroviral therapy and exhibited diverse responses to treatment as determined by viral load assay and percentage of CD4⁺ cells (FIG. 11A). The procedure used to prepare PBMCs and CD4⁺ T-cells, examination of CD4⁺ T-cells, and the timeline for lentivirus treatments and cell harvest are shown in FIG. 16B. The purity of the CD4⁺ T-cells was confirmed by flow cytometry of FITC conjugated anti-CD4 antibody (FIG. 16C).

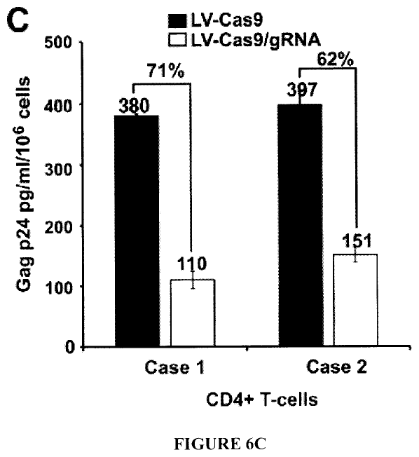

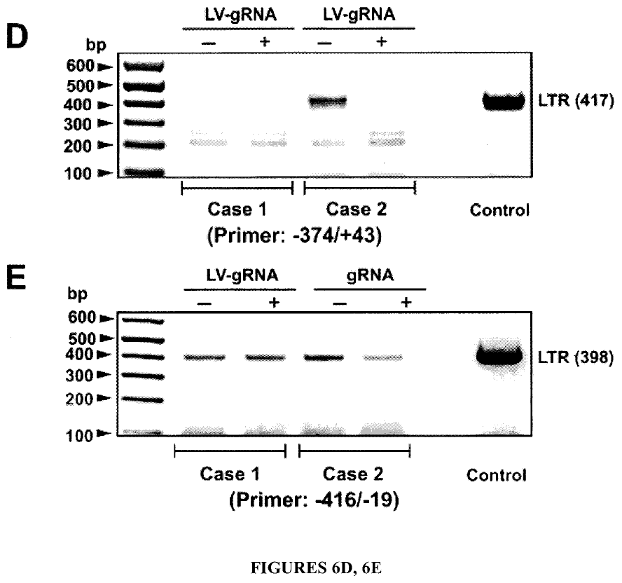

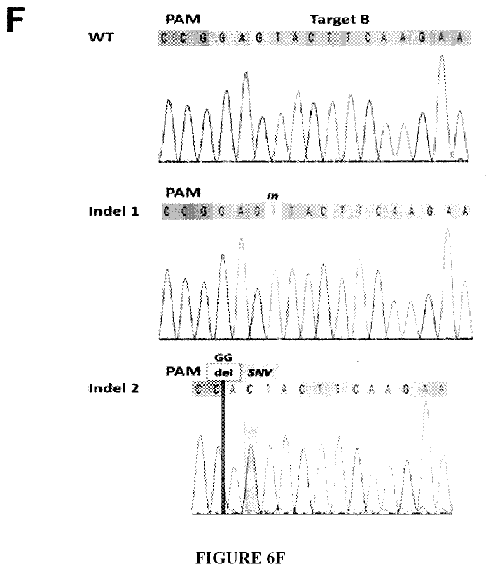

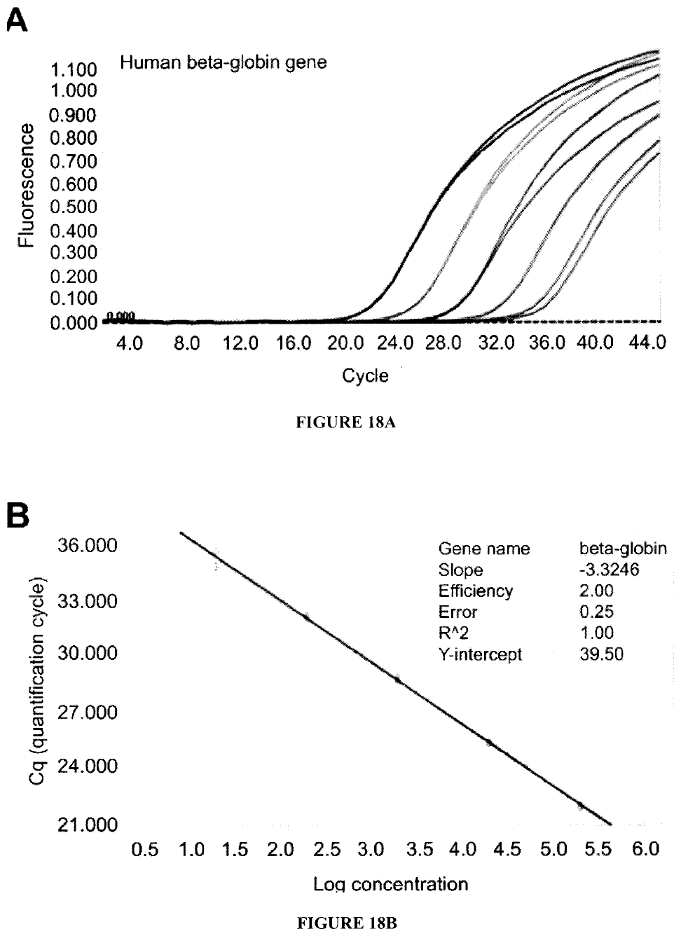

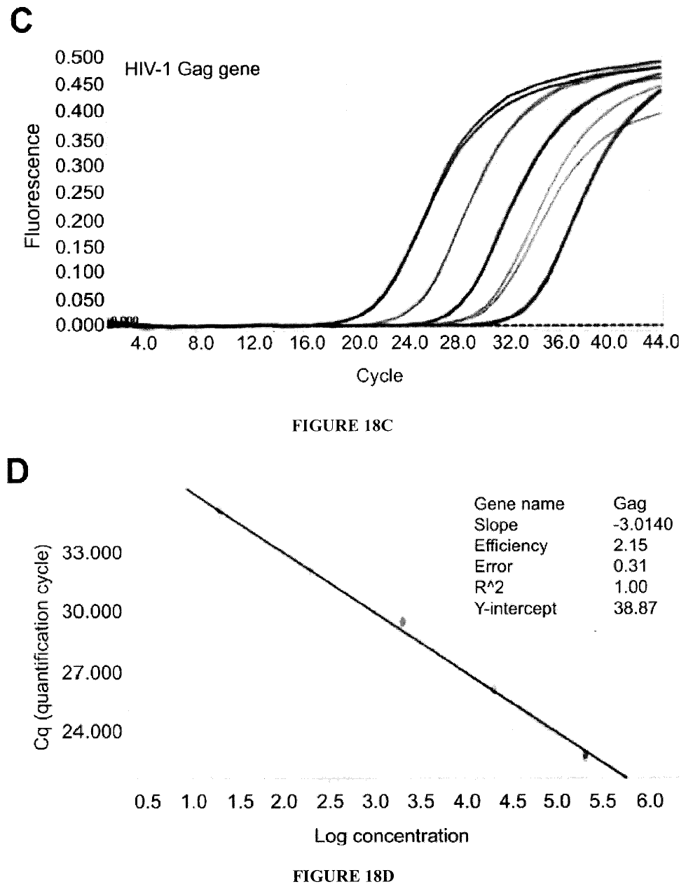

Results of transducing PBMC's with lentivirus-Cas9 and lentivirus-Cas9/gRNA revealed a substantial decrease, 81% in Case 1 and 91% in Case 2, in the viral copy number of cell populations expressing Cas9 and gRNA (FIG. 6A). Similar results were obtained after lentiviral transduction of CD4⁺ T-cells, which showed >92% reduction in viral copies in Case 1 and 56% for Case 2 upon expression of both Cas9 and gRNA, compared to control cells expressing only Cas9 (FIG. 6B). Standard curves and amplification plots served for absolute quantification of β-globin and Gag gene copy number is shown in FIGS. 18A-18D. Examination of Gag p24 gene production in the CD4⁺ T-cells confirmed viral replication was decreased in Case 1 (71%) and Case 2 (62%) upon single transduction of the cells with lentivirus-Cas9/gRNA compared to that seen with lentivirus-Cas9 (FIG. 6C). Also, the level of Gag p24 was examined in PBMCs obtained from Cases 3 and 4 after delivery of Cas9/gRNA by lentivirus. Results from this study showed 39% and 54% decrease in HIV-1 p24 production from Cases 3 and 4, respectively, after transduction of the cells with therapeutic lentivirus (FIG. 17).

Next, the nature of mutations introduced by Cas9/gRNAs in the patient samples was assessed by amplifying and sequencing the viral DNA. The initial gene amplification of the CD4⁺ T-cells using primers spanning −374/+43 failed to detect any band in Case 1 and in Case 2 a DNA band was observed in the control sample that lacked gRNA expression (FIG. 6D). This observation evidences that the HIV-1 genome sequence in case 1 may differ from those of the primers that were used for gene amplification (FIG. 6D). In case 2, where the expected DNA fragment was detected in untreated cells, the mutations that were introduced by Cas9/gRNA may have eliminated the recognition of DNA sequence by the PCR primer, thus interfering with DNA amplification. The use of an alternative set of primers that recognizes different regions of the LTR led to production of the expected 398-nucleotide amplicon in all samples (FIG. 6E). It is possible that similar to the results from 2D10 cells after treatment with Cas9/gRNAs (shown in FIG. 2A), some of the 397 nucleotide DNA fragments seen in the presence of gRNA expression result from joining of the remaining 5′ and 3′ sequences of the viral LTR after excision of the entire HIV-1 coding sequence. Sequencing of the amplicon verified the effect of Cas9/gRNA on editing of the viral genome at the expected positions and showed the presence of InDel and single nucleotide variation (SNV) mutations within and/or next to the PAM sequence within the LTR (FIG. 6F).

Table 2 shows the mapping rate and coverage.

TABLE 2

| Sample | +Cas9/+gRNA | +Cas9/−gRNA |
|---|---|---|
| Total | 2304621804 (100%) | 2153253838 (100%) |
| Duplicate | 42615862 (19.60%) | 326664344 (15.50%) |
| Mapped | 2175107441 (94.38%) | 2108094482 (97.90%) |
| Properly mapped | 2133746358 (92.59%) | 2057204364 (95.54%) |
| PE mapped | 2173896582 (94.33%) | 2107021448 (97.85%) |
| SE mapped | 2421718 (0.11%) | 2146068 (01.10%) |
| With mate mapped to a | 930716 (0.41%) | 9569940 (0.33%) |
| different chr | | |
| With mate mapped to a | 6944857 (0.30%) | 7044381 (0.33%) |
| different chr (mapQ >= 5) | | |
| Average sequencing depth | 112.72 | 109.25 |
| Coverage | 99.67% | 99.69% |
| Coverage at least 4X | 99.48% | 99.51% |
| Coverage at least 10X | 99.00% | 99.08% |
| Coverage at least 20X | 97.29% | 97.57% |

Total: The number of total clean rads
Duplicate: The number of duplication reads
Mapped: the number of total reads that mapped to thge reference genome (percentage)
Properly mapped: The number of reads that mapped to the reference genome and the direction is right
PE mapped: The number of pair-end reads that mapped to the reference genome (percentage)
SE mapped: The number of single-end reads that mapped to the reference genome
With mate mapped to a different chr: The number of mate reads that mapped to the different chromosomes (percentage)
With mate mapped to a diofferent chr (mapQ => 5): The number of mate reads that mapped to the different chromosomes and thw MAQ > 5
Average sequencing depth: The average sequencing depth that mapped to the reference genome
Coverage: The sequence coverage of the genome
Coverage at least 4X: The percentage of bases with depth >4X in whole genome bases
Coverage at least 10X: The percentage of bases with depth >10X in whole genome bases
Coverage at least 20X: The percentage of bases with depth >20X in whole genome bases

Table 3 shows the distribution of Insertion/Deletions (InDels) in different genomic regions.

TABLE 3

| | +Cas9/ | +Cas9/ | +Cas9/+gRNA over— |
|---|---|---|---|
| Sample | +gRNA | −gRNA | +Cas9/−gRNA |
| CDS | 1701 | 1746 | 164 |
| frameshift_deletion | 866 | 910 | 124 |
| frameshift_insertion | 279 | 275 | 33 |
| nonframeshift_deletion | 235 | 232 | 2 |
| nonframeshift_insertion | 187 | 196 | 0 |
| stopgain | 16 | 11 | 1 |
| stoploss | 1 | 1 | 0 |
| unknown | 117 | 121 | 4 |
| Intronic | 537492 | 538344 | 12229 |
| UTR3 | 12154 | 12144 | 354 |
| UTR5 | 1450 | 1446 | 58 |
| Splicing | 498 | 500 | 20 |
| ncRNA_exonic | 2638 | 2629 | 78 |
| ncRNA_intronic | 71426 | 71581 | 1758 |
| ncRNA_UTR3 | 389 | 388 | 16 |
| ncRNA_UTR5 | 45 | 50 | 2 |
| ncRNA_splicing | 80 | 74 | 1 |
| upstream | 9229 | 9256 | 209 |
| downstream | 10296 | 10204 | 199 |
| intergenic | 711001 | 712949 | 17310 |
| Total | 1358399 | 1361311 | 32399 |

Note Sample: Sample name
CDS: the number of InDel in exonic region
frameshift_deletion: a deletion of one or more nucleotides that cause frameshift changes in protein coding sequence. The deletion length is not multiple of 3.
frameshift_insertion: an insertion of one or more mucleotides that cause frameshift changes in protein coding sequence. The insertion is not multpile of 3.
nonframeshift_deletion: non-framesgift deletion, does not change coding protein frame deletion, the deletion length is multiple of 3.
nonframeshift_insertion: non-frameshift insertion, does not change coding protein frame deletion, the deletion length is multiple of 3.
stopgain: frameshift insertion/deletion, nonframeshift insertion/deletion or block substitution that lead to the immediate creation of stop codon at the variant site.
stoploss: frameshift insertion/deletion, nonframeshift insertion/deletion or block substitution that lead to the immediate elimination of stop codon at the variant site.
unknown: unknown function (due to various errors in the gene structure definition in the database file.
intronic: the number of InDel in intonic region
UTR3: the number of InDel in 3′ UTR region
UTR5: the number of InDel in 5′ UTR region
splicing: the number of InDel in 4bp splicing junction region
ncRNA_exonic: the number of InDel in non-coding RNA exonic region
ncRNA_intronic: the number of InDel in non-coding RNA intronic region
ncRNA_UTR3: the number of InDel in 3′UTR of non-coding RNA
ncRNA_UTR5: the number of InDel in 5′UTR of non-coding RNA
ncRNA_splicing: the number of InDel in 4bp splicing junction of non-coding RNA
upstream: the number of InDel in the 1 kb upstream region of transcription start site
downstream: the number of InDel in the 1 kb downstream region of transcription ending site
intergenic: the number of InDel in the intergenic region
Total: the total number of InDel

TABLE 4
list the distribution of Single Nucleotide Polymorphisms (SNP) in
different genomic regions

| | | | +Cas9/+gRNA over— |
|---|---|---|---|
| Sample | +Cas9/+gRNA | +Cas9/−gRNA | +Cas9/−gRNA |
| CDS | 29643 | 29982 | 1085 |
| synonymous_SNP | 13747 | 13894 | 347 |
| missense_SNP | 14924 | 15091 | 679 |
| stopgain | 378 | 394 | 43 |
| stoploss | 15 | 15 | 0 |
| unknown | 579 | 588 | 16 |
| Intronic | 1369993 | 1374507 | 17921 |
| UTR3 | 28450 | 28628 | 564 |
| UTR5 | 6626 | 6659 | 193 |
| Splicing | 845 | 882 | 32 |
| ncRNA_exonic | 12827 | 12918 | 175 |
| ncRNA_intronic | 206923 | 207516 | 2352 |
| ncRNA_UTR3 | 809 | 824 | 11 |
| ncRNA_UTR5 | 164 | 171 | 5 |
| ncRNA_splicing | 139 | 141 | 2 |
| upstream | 26386 | 26577 | 493 |
| downstream | 25707 | 25782 | 403 |
| intergenic | 2252883 | 2258511 | 23378 |
| Total | 3961395 | 3973098 | 46614 |

Note Sample: Sample name
CDS: the number of InDel in exonic region
synonymopus_SNP: a single nucleotide change that does not cause an amino acid change
missense_SNP: a single nucleotide change that causes an amino acid change
stopgain: a nonsynonymous SNP that leads to the immediate creation of stop codon at the variant site
stoploss: a nonsynonymous SMP that leads to the immediate elimination of stop codon at the variant site.
unknown: unknown function (due to various errors in the gene structure definition in the database file).
intronic: the number of Somatic SNP in intronic region
UTR3: the number of Somatic SNP in 3′ UTR region
UTR5: the number of Somatic SNP in 5′ UTR region
intergenic: the number of Somatic SNP in the intergenic region
ncRNA_exonic: the number of Somatic SNP in non-coding RNA exonic region
ncRNA_intronic: the number of Somatic SNP in onoc-coding RNA intronic region
upstream: the number of Somatic SNP in the 1 kb upstream region of transcription start site
downstream: the number of Somatic SNP in the 1 kb downstream region of transcription ending dite
splicing: the number of Somatic SNP in 10bp splicing junction region
ncRNA_UTR3: the number of Somatic SNP in 3′UTR of non-coding RNA
ncRNA_UTR5: the number of Somatic SNP 5′UTR of non-coding RNA
ncRNA_splicing: the number of Somatic SNP in 10bp splicing junction of non-coding RNA
Total: the total number of Somatic SNP

TABLE 5

| | | | | Post | | | | | |
|---|---|---|---|---|---|---|---|---|---|
| | Total | Somatic | | Homopolymeric | | | | | |
| | InDels | InDels^{a} | Post dbSNP Filter | Filter | Total SNVs | Somatic SNVs^{b} | Post dbSNP Filter | Total SVs^{c} | Somatic SVs^{d} |
| +Cas9/−gRNA | 1361311 | | | | 3973098 | | | 3433 | |
| +Cas9/+gRNA | 1358399 | 32399 | 30156 | 989 | 3961395 | 46614 | 43848 | 3487 | 52 |

^{a}Somatic InDels—means the specific InDels in +Cas9/+gRNA compared to control cell lines called by Strelka.
^{b}Somatic SNVs—means the specific SNVs in +Cas9/+gRNA compared to control cell lines called by MuTect.
^{c}Total SV—onlyincludes the SV types of deletion and insertion called by Crest
^{d}Somatic SVs—means the specific SVs (deletion and insertion) in +Cas9/+gRNA comparted to control cell lines called by Crest

Discussion

In summary, the results show that lentivirally-delivered Cas9/gRNAs A/B significantly decreased viral copy numbers and protein levels in PBMCs and CD4⁺ T-cells from HIV-1 infected patients. PCR with primer sets directed within the LTR i0 amplified and detected residual viral DNA fragments that were not completely deleted in these cells, yet were affected by Cas9/gRNAs and contained InDel mutants near the PAM sequence. These findings verified that CRISPR/Cas9 exerted efficacious antiviral activity in the PBMCs of HIV-1 patients.

ART treatment is unable to eradicate HIV-1 from infected patients who must therefore undergo life-long treatment. The new therapeutic strategy described herein, will achieve permanent remission allowing patients to stop ART and reduce its attendant costs and potential long-term side effects. The developed CRISPR/Cas9 techniques that eradicated integrated copies of HIV-1 from human CD4⁺ T-cells, inhibited HIV-1 infection in primary cultured human CD4+ T-cells, and suppressed viral replication ex vivo in peripheral blood mononuclear cells (PBMCs) and CD4+ T-cells of HIV-1+ patients. They also address a further key issue, providing evidence that such gene editing effectively impedes viral replication without causing genotoxicity to host DNA or eliciting destructive effects via host cell pathways. In this study, as a first step, the clonal 2D10 cell line was used as a human T-cell latency model to establish: (i) the ability of Cas9/gRNA in removing the entire coding sequence of the integrated copies of the HIV-1 DNA using ultradeep whole genome sequencing and (ii) investigate its safety related to off-target effects and cell viability. Once these goals were accomplished, the study shifted attention to primary cell cultures as well as patient samples to examine the efficiency of the CRISPR/Cas9 in affecting viral DNA load in a laboratory setting.

It was found that CRISPR/Cas9 edited multiple copies of viral DNA scattered among the chromosomes. Combined treatment of latently-infected T cells with Cas9 plus gRNAs A and B that recognize specific DNA motifs within the LTR U3 region efficiently eliminated the entire viral DNA fragment spanning between the two LTRs. The remaining 5′ LTR and 3′ LTR cleavage sites by Cas9 and gRNA B in chromosome 1, and by Cas9 and gRNAs A and B in chromosome 16, were joined by host DNA repair at sites located precisely three nucleotides upstream of the PAM. Genome-wide assessment of CRISPR/Cas9-treated HIV-1-infected 2D10 cells clearly verified complete excision of the integrated copies of viral DNA from the second intron of RSBN1 and exon 2 of MSRB1 genes. To address the specificity and potential off-target and adverse effects, a comprehensive analysis at an unprecedented level of detail was conducted, by whole-genome sequencing and bioinformatic analyses. These revealed many naturally-occurring mutations in the genomes of control cells and gRNAs A- and B-mediated HIV-1 DNA eradication. The mutations discovered included naturally-occurring InDels, base excisions, and base substitutions, all of which are, more or less, expected in rapidly growing cells in culture, including Jurkat 2D10 cells. The critical issue is the discovery herein that none of these mutations resulted from the gene-editing system, as no sequence identities were identified with either gRNA A or B within 1200 nucleotides of any such mutation site. Further, this method for HIV-1 DNA excision had no adverse effects on proximal or distal cellular genes and showed no impact on cell viability, cell cycle progression or proliferation, and did not induce apoptosis, thus strongly supporting its safety at this translational phase, by all in vitro measures assessed in cultured cells. It was found that the expression levels of Cas9 and the gRNAs diminished after several passages and eventually disappeared, but as long as Cas9 and single or multiplex gRNAs were present, cells remained protected against new HIV-1 infection.

Another key translational feasibility question that was addressed was whether CRISPR/Cas9-mediated HIV-1 eradication can prevent or suppress HIV-1 infection in the most relevant human and patient target cell populations. It was found that in PBMCs and CD4⁺ T-cells from HIV-1 infected patients that lentivirally-delivered Cas9/gRNAs A/B significantly decreased viral copy numbers and protein levels. Using primer sets directed within the LTR, residual viral DNA fragments that were amplified and detected were not completely deleted in these cells, yet were affected by Cas9/gRNAs and contained InDel mutants near the PAM sequence. These findings verified that CRISPR/Cas9 exerted efficacious antiviral activity in the PBMCs of HIV-1 patients. It was also found that introducing Cas9/gRNAs A/B via lentiviral delivery into primary cultured human CD4⁺ HIV-1_{JRFL}- or HIV-1_{NL4_3}-infected T-cells significantly reduced viral copy numbers, corroborating that stably-integrated HIV-1-directed Cas9 and gRNAs (distinct from the gRNAs A and B used presently) conferred resistance to HIV-1 infection in cell lines. With the notion that CRISPR/Cas9 can target both integrated, as well as episomal DNA sequences, as evidenced by its editing ability of various human viruses as well as plasmid DNAs in either configuration, it is likely that both the integrated as well as pre-integrated, free-floating intracellular HIV-1 DNA are edited by Cas9/gRNA.

As noted, during the course of these studies no ART was included prior to the treatment with CRISPR/Cas9 as the goal in this study was to determine the extent of viral suppression during the productive stage of viral infection. A significant level of suppression was observed, providing evidence that CRISPR/Cas9 effectively disabled expression of the functionally active integrated copies of HIV-1 DNA in the host chromosome. This notion is supported by the observations using 2D10 CD4⁺ T-cells where the latent copies of HIV-1 that are integrated in chromosomes 1 and 16 were effectively eliminated by CRISPR/Cas9. In conclusion, the findings herein, show comprehensively and conclusively that the entire coding sequence of host-integrated HIV-1 was eradicated in human T cells, providing strong support for the translatability of such a system to T-cell-directed HIV-1 therapies in patients.

The invention has been described in an illustrative manner, and it is to be understood that the terminology that has been used is intended to be in the nature of words of description rather than of limitation. Obviously, many modifications and variations of the present invention are possible in light of the above teachings. It is, therefore, to be understood that within the scope of the appended claims, the invention can be practiced otherwise than as specifically described.

What is claimed is:

1. A composition for use in inactivating a proviral DNA integrated into the genome of a host cell latently infected with human immunodeficiency virus (HIV), the composition comprising:
at least one isolated nucleic acid sequence encoding a Clustered Regularly Interspaced Short Palindromic Repeat (CRISPR)-associated endonuclease, and
at least one guide RNA (gRNA), said at least one gRNA having a spacer sequence that is complementary to a target sequence in a long terminal repeat (LTR) of a proviral HIV DNA.

2. The composition according to claim 1, wherein said at least one gRNA comprises a nucleic acid sequence complementary to a target nucleic acid sequence having a sequence identity of at least 75% to one or more SEQ ID NOS: 1 to 66, fragments, mutants, variants or combinations thereof.

3. The composition according to claim 1, wherein said at least one gRNA comprises a nucleic acid sequence having a sequence identity of at least 75% to one or more SEQ ID NOS: 1 to 66, fragments, mutants, variants or combinations thereof.

4. The composition according to claim 1, wherein said at least one gRNA comprises at least one nucleic acid sequence complementary to a target nucleic acid sequence comprising SEQ ID NOS: 1 to 66, fragments, mutants, variants or combinations thereof.

5. The composition according to claim 1, wherein said at least one gRNA comprises at least one nucleic acid sequence comprising SEQ ID NOS: 1 to 66, fragments, mutants, variants or combinations thereof.

6. The composition according to claim 1, wherein said at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

7. A method of inactivating a proviral human immunodeficiency virus (HIV) DNA integrated into the genome of a host cell latently infected with HIV, including the steps of:
treating the host cell with a composition comprising a Clustered Regularly Interspaced Short Palindromic Repeat (CRISPR)-associated endonuclease, and at least one guide RNA (gRNA) having a spacer sequence that is complementary to a target sequence in a long terminal repeat (LTR) of a proviral HIV DNA; and
inactivating the proviral DNA.

8. The method according to claim 7, wherein said at least one gRNA comprises a nucleic acid sequence complementary to a target nucleic acid sequence having a sequence identity of at least 75% to one or more SEQ ID NOS: 1 to 66, fragments, mutants, variants or combinations thereof.

9. The method according to claim 7, wherein said at least one gRNA comprises a nucleic acid sequence having a sequence identity of at least 75% to one or more SEQ ID NOS: 1 to 66, fragments, mutants, variants or combinations thereof.

10. The method of claim 7, wherein said at least one gRNA comprises at least one nucleic acid sequence complementary to a target nucleic acid sequence comprising SEQ ID NOS: 1 to 66, fragments, mutants, variants or combinations thereof.

11. The method according to claim 7, wherein said at least one gRNA comprises at least one nucleic acid sequence comprising SEQ ID NOS: 1 to 66, fragments, mutants, variants or combinations thereof.

12. The method according to claim 7, wherein the at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

13. A lentiviral expression vector composition for use in inactivating proviral DNA integrated into the genome of a host cell latently infected with human immunodeficiency virus (HIV), including:
an isolated nucleic acid encoding a Clustered Regularly Interspaced Short Palindromic Repeat (CRISPR)-associated endonuclease, and at least one isolated nucleic acid encoding at least one guide RNA (gRNA) including a spacer sequence that is complementary to a target sequence in a long terminal repeat (LTR) of a proviral HIV DNA,
said CRISPR-associated endonuclease and said at least one gRNA being included in at least one lentiviral expression vector,
wherein said at least one lentiviral expression vector induces the expression of said CRISPR-associated endonuclease and said at least one gRNA in a host cell.

14. The lentiviral expression vector composition according to claim 13, wherein said at least one gRNA comprises a nucleic acid sequence complementary to a target nucleic acid sequence having a sequence identity of at least 75% to one or more SEQ ID NOS: 1 to 66, fragments, mutants, variants or combinations thereof.

15. The lentiviral expression vector composition of claim 13, wherein said at least one gRNA comprises a nucleic acid sequence having a sequence identity of at least 75% to one or more SEQ ID NOS: 1 to 66, fragments, mutants, variants or combinations thereof.

16. The lentiviral expression vector composition according to claim 13, wherein said at least one gRNA comprises at least one nucleic acid sequence complementary to a target nucleic acid sequence comprising SEQ ID NOS: 1 to 66, fragments, mutants, variants or combinations thereof.

17. The lentiviral expression vector composition according to claim 13, wherein said at least one gRNA comprises at least one nucleic acid sequence comprising SEQ ID NOS: 1 to 66, fragments, mutants, variants or combinations thereof.

18. The lentiviral expression vector composition according to claim 13, wherein said at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

19. The lentiviral expression vector composition according to claim 13, wherein said CRISPR associated endonuclease and said at least one gRNA are incorporated into in a single lentiviral expression vector.

20. The lentiviral expression vector composition according to claim 13, wherein said CRISPR associated endonuclease and said at least one gRNA are incorporated into separate lentiviral expression vectors.

21. A method of eliminating a proviral DNA integrated into the genome of ex vivo cultured host cells latently infected with human immunodeficiency virus (HIV), including the steps of:
obtaining a population of host cells latently infected with HIV, wherein a proviral HIV DNA is integrated into the host cell genome;
culturing the host cells ex vivo;
treating the host cells with a composition comprising a Clustered Regularly Interspaced Short Palindromic Repeat (CRISPR)-associated endonuclease, and at least one guide RNA (gRNA), the at least one gRNA having a spacer sequence that is complementary to a target sequence in a long terminal repeat (LTR) of the proviral HIV DNA; and
eliminating the proviral DNA from the host cell genome.

22. The method according to claim 21, wherein said step of obtaining a population of host cells is further defined as obtaining a population of human host cells.

23. The method according to claim 21, wherein said step of obtaining a population of host cells is further defined as obtaining a population of human peripheral blood mononuclear cells, or obtaining a population of CD4⁺ T cells.

24. The method according to claim 21, wherein said at least one gRNA comprises a nucleic acid sequence complementary to a target nucleic acid sequence having a sequence identity of at least 75% to one or more SEQ ID NOS: 1 to 66, fragments, mutants, variants or combinations thereof.

25. The method according to claim 21, wherein said at least one gRNA comprises a nucleic acid sequence having a sequence identity of at least 75% to one or more SEQ ID NOS: 1 to 66, fragments, mutants, variants or combinations thereof.

26. The method of claim 21, wherein said at least one gRNA comprises at least one nucleic acid sequence complementary to a target nucleic acid sequence comprising SEQ ID NOS: 1 to 66, fragments, mutants, variants or combinations thereof.

27. The method according to claim 21, wherein said at least one gRNA comprises at least one nucleic acid sequence comprising SEQ ID NOS: 1 to 66, fragments, mutants, variants or combinations thereof.

28. The method according to claim 21, wherein the at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

29. The method according to claim 21, wherein said treating step is further includes the step of expressing, in the latently infected T cells, the CRISPR-associated endonuclease, and the at least one guide RNA (gRNA).

30. A method of treating a patient having a latent human immunodeficiency virus (HIV) infection of T cells, including the steps of:
obtaining from the patient a population including latently infected T cells, wherein a proviral HIV DNA is integrated into the T cell genome;
culturing the latently infected T cells ex vivo;
treating the latently infected T cells with a composition comprising a Clustered Regularly Interspaced Short Palindromic Repeat (CRISPR)-associated endonuclease, and at least one guide RNA (gRNA), the at least one gRNA having a spacer sequence that is complementary to a target sequence in a long terminal repeat (LTR) of a proviral DNA;
eliminating the integrated proviral HIV DNA from the T cell genome;
producing an HIV-eliminated T cell population;
infusing the HIV-eliminated T cell population into the patient; and
treating the patient.

31. The method according to claim 30, wherein said step of obtaining a population including latently infected T cells is further defined as obtaining a population of human peripheral blood mononuclear cells or, obtaining a population of CD4⁺ T cells.

32. The method according to claim 30, wherein said at least one gRNA comprises a nucleic acid sequence complementary to a target nucleic acid sequence having a sequence identity of at least 75% to one or more SEQ ID NOS: 1 to 66 or combinations thereof.

33. The method according to claim 30, wherein said at least one gRNA comprises a nucleic acid sequence having a sequence identity of at least 75% to one or more SEQ ID NOS: 1 to 66, fragments, mutants, variants or combinations thereof.

34. The method of claim 30, wherein said at least one gRNA comprises at least one nucleic acid sequence complementary to a target nucleic acid sequence comprising SEQ ID NOS: 1 to 66, fragments, mutants, variants or combinations thereof.

35. The method according to claim 30, wherein said at least one gRNA comprises at least one nucleic acid sequence comprising SEQ ID NOS: 1 to 66, fragments, mutants, variants or combinations thereof.

36. The method according to claim 30, wherein the at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

37. The method according to claim 30 wherein said treating step is further includes the step of expressing in, the latently infected T cells, the CRISPR-associated endonuclease, and the at least one gRNA.

38. A method of preventing human immunodeficiency virus (HIV) infection of T cells of a patient at risk of HIV infection, including the steps of:
determining that a patient is at risk of HIV infection;
exposing T cells of the patient at risk of HIV1 infection to an effective amount of an expression vector composition including an isolated nucleic acid encoding a Clustered Regularly Interspaced Short Palindromic Repeat (CRISPR)-associated endonuclease, and at least one isolated nucleic acid encoding at least one guide RNA (gRNA) including a spacer sequence that is complementary to a target sequence in the an LTR of HIV1 DNA;
stably expressing the CRISPR-associated endonuclease and the at least one gRNA in the T cells; and
preventing HIV infection of the T cells.

39. The method according to claim 38, wherein said step of exposing the T cells is further defined as exposing the T cells in vivo.

40. The method according to claim 38, wherein said step of exposing the T cells is further defined as exposing the T cells ex vivo, and said step of stably expressing is followed by the step of infusing the T cells into the patient.

41. The method according to claim 38, wherein the expression vector composition is a lentiviral vector composition.

42. The method according to claim 38, wherein said at least one gRNA comprises a nucleic acid sequence complementary to a target nucleic acid sequence having a sequence identity of at least 75% to one or more SEQ ID NOS: 1 to 66 or combinations thereof.

43. The method according to claim 38, wherein said at least one gRNA comprises a nucleic acid sequence having a sequence identity of at least 75% to one or more SEQ ID NOS: 1 to 66, fragments, mutants, variants or combinations thereof.

44. The method of claim 38, wherein said at least one gRNA comprises at least one nucleic acid sequence complementary to a target nucleic acid sequence comprising SEQ ID NOS: 1 to 66, fragments, mutants, variants or combinations thereof.

45. The method according to claim 38, wherein said at least one gRNA comprises at least one nucleic acid sequence comprising SEQ ID NOS: 1 to 66, fragments, mutants, variants or combinations thereof.

46. The method according to claim 38, wherein the at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

47. A pharmaceutical composition for the eradication of integrated HIV-1 DNA in the cells of a mammalian subject, including an isolated nucleic acid sequence encoding a Clustered Regularly Interspaced Short Palindromic Repeat (CRISPR)-associated endonuclease; at least one isolated nucleic acid sequence encoding at least one guide RNA (gRNA) that is complementary to a target sequence in a long terminal repeat (LTR) of a proviral HIV-1 DNA; said isolated nucleic acid sequences being included in at least one expression vector.

48. The pharmaceutical composition according to claim 47, wherein said at least one gRNA comprises a nucleic acid sequence complementary to a target nucleic acid sequence having a sequence identity of at least 75% to one or more SEQ ID NOS: 1 to 66, fragments, variants, mutants or combinations thereof.

49. The pharmaceutical composition according to claim 47, wherein said at least one gRNA comprises a nucleic acid sequence having a sequence identity of at least 75% to one or more SEQ ID NOS: 1 to 66, fragments, mutants, variants or combinations thereof.

50. The pharmaceutical composition according to claim 47, wherein said at least one gRNA comprises at least one nucleic acid sequence complementary to a target nucleic acid sequence comprising SEQ ID NOS: 1 to 66, fragments, mutants, variants or combinations thereof.

51. The pharmaceutical composition according to claim 47, wherein said at least one gRNA comprises at least one nucleic acid sequence comprising SEQ ID NOS: 1 to 66, fragments, mutants, variants or combinations thereof.

52. The pharmaceutical composition according to claim 47, wherein the at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

53. The pharmaceutical composition according to claim 47, wherein said expression vector is a lentiviral vector.

54. A method of treating a mammalian subject infected with HIV-1, including the steps of: determining that a mammalian subject is infected with HIV-1, administering, to the subject, an effective amount of a pharmaceutical composition according to claim 47; and treating the subject for HIV-1 infection.

55. An isolated nucleic acid encoding a Clustered Regularly Interspaced Short Palindromic Repeat (CRISPR)-associated endonuclease, and/or at least one isolated nucleic acid encoding at least one guide RNA (gRNA) including a spacer sequence that is complementary to a target sequence in a long terminal repeat (LTR) of a proviral HIV DNA,
said CRISPR-associated endonuclease and said at least one gRNA being included in at least one expression vector,
wherein said at least one expression vector induces the expression of said CRISPR-associated endonuclease and said at least one gRNA in a host cell.

56. The isolated nucleic acid sequence according to claim 55, wherein said at least one gRNA comprises a nucleic acid sequence complementary to a target nucleic acid sequence having a sequence identity of at least 75% to one or more SEQ ID NOS: 1 to 66, fragments, variants, mutants or combinations thereof.

57. The isolated nucleic acid sequence according to claim 55, wherein said at least one gRNA comprises a nucleic acid sequence having a sequence identity of at least 75% to one or more SEQ ID NOS: 1 to 66, fragments, mutants, variants or combinations thereof.

58. The isolated nucleic acid sequence according to claim 55, wherein said at least one gRNA comprises at least one nucleic acid sequence complementary to a target nucleic acid sequence comprising SEQ ID NOS: 1 to 66, fragments, mutants, variants or combinations thereof.

59. The isolated nucleic acid sequence according to claim 55, wherein said at least one gRNA comprises at least one nucleic acid sequence comprising SEQ ID NOS: 1 to 66, fragments, mutants, variants or combinations thereof.

60. The isolated nucleic acid sequence according to claim 55, wherein said at least one gRNA is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

61. The isolated nucleic acid sequence according to claim 55, wherein said CRISPR associated endonuclease and said at least one gRNA are incorporated into in a single lentiviral expression vector.

62. The isolated nucleic acid sequence according to claim 55, wherein said CRISPR associated endonuclease and said at least one gRNA are incorporated into separate expression vectors.

63. A kit for the treatment or prophylaxis of HIV-1 infection, including a measured amount of a composition comprising at least one isolated nucleic acid sequence encoding a Clustered Regularly Interspaced Short Palindromic Repeat (CRISPR)-associated endonuclease, and at least one nucleic acid sequence encoding one or more guide RNAs (gRNAs), wherein each of said one or more gRNAs includes a spacer sequence complementary to a target sequence in a long terminal repeat (LTR) of an HIV-1 provirus; and one or more items selected from the group consisting of packaging material, a package insert comprising instructions for use, a sterile fluid, a syringe and a sterile container.

64. The kit according to claim 63, wherein said one or more gRNAs is selected from gRNA A, having a spacer sequence complementary to a target sequence SEQ ID NO: 1 or to a target sequence SEQ ID NO: 2 in the proviral DNA; gRNA B, having a spacer sequence complementary to a target sequence SEQ ID NO: 3 or to a target sequence SEQ ID NO: 4 in the proviral DNA; or combination of gRNA A and gRNA B.

65. The kit according to claim 63, wherein said at least one of said isolated nucleic acid sequences is included in an expression vector.

66. The kit according to claim 65, wherein said expression vector is a lentiviral expression vector.

67. An isolated nucleic acid sequence comprising one or more nucleic acid sequences having at least a 75% sequence identity to any one or more of SEQ ID NOS: 1 to 66, fragments, variants, mutants or combinations thereof.

68. The isolated nucleic acid sequence comprising any one or more of SEQ ID NOS: 1 to 66, fragments, variants, mutants or combinations thereof.


SEQUENCE LISTING (102 sequences)

SEQ ID NO: 1 - DNA, 30 bp, Artificial Sequence
  Description of Artificial Sequence Synthetic oligonucleotide
agggccagggatcagatatccactgacctt

SEQ ID NO: 2 - DNA, 20 bp, Artificial Sequence
  Description of Artificial Sequence Synthetic oligonucleotide
atcagatatccactgacctt

SEQ ID NO: 3 - DNA, 30 bp, Artificial Sequence
  Description of Artificial Sequence Synthetic oligonucleotide
agctcgatgtcagcagttcttgaagtactc

SEQ ID NO: 4 - DNA, 20 bp, Artificial Sequence
  Description of Artificial Sequence Synthetic oligonucleotide
cagcagttcttgaagtactc

SEQ ID NO: 5 - DNA, 33 bp, Artificial Sequence
  Description of Artificial Sequence Synthetic oligonucleotide
agggccagggatcagatatccactgacctttgg

SEQ ID NO: 6 - DNA, 33 bp, Artificial Sequence
  Description of Artificial Sequence Synthetic oligonucleotide
agctcgatgtcagcagttcttgaagtactccgg

SEQ ID NO: 7 - DNA, 24 bp, Artificial Sequence
  Description of Artificial Sequence Synthetic oligonucleotide
tggaagggctaattcactcccaac

SEQ ID NO: 8 - DNA, 24 bp, Artificial Sequence
  Description of Artificial Sequence Synthetic oligonucleotide
ttagcagaactacacaccagggcc

SEQ ID NO: 9 - DNA, 24 bp, Artificial Sequence
  Description of Artificial Sequence Synthetic oligonucleotide
ccgagagctcccaggctcagatct

SEQ ID NO: 10 - DNA, 23 bp, Artificial Sequence
  Description of Artificial Sequence Synthetic oligonucleotide
gatctgtggatctaccacacaca

SEQ ID NO: 11 - DNA, 23 bp, Artificial Sequence
  Description of Artificial Sequence Synthetic oligonucleotide
gctgcttatatgtagcatctgag

SEQ ID NO: 12 - DNA, 28 bp, Artificial Sequence
  Description of Artificial Sequence Synthetic oligonucleotide
cgccaagcttgaataggagctttgttcc

SEQ ID NO: 13 - DNA, 30 bp, Artificial Sequence
  Description of Artificial Sequence Synthetic oligonucleotide
ctaggatccaggagctgttgatcctttagg

SEQ ID NO: 14 - DNA, 23 bp, Artificial Sequence
  Description of Artificial Sequence Synthetic oligonucleotide
gtggactttggatggtgagatag

SEQ ID NO: 15 - DNA, 23 bp, Artificial Sequence
  Description of Artificial Sequence Synthetic oligonucleotide
gcctggcaagagtgaactgagtc

SEQ ID NO: 16 - DNA, 23 bp, Artificial Sequence
  Description of Artificial Sequence Synthetic oligonucleotide
aagataatgagttgtggcagagc

SEQ ID NO: 17 - DNA, 24 bp, Artificial Sequence
  Description of Artificial Sequence Synthetic oligonucleotide
tctacctggtaatccagcatctgg

SEQ ID NO: 18 - DNA, 23 bp, Artificial Sequence
  Description of Artificial Sequence Synthetic oligonucleotide
ataggaggaaggcaccaagaggg

SEQ ID NO: 19 - DNA, 23 bp, Artificial Sequence
  Description of Artificial Sequence Synthetic oligonucleotide
aatgatgctttggtcctactcct

SEQ ID NO: 20 - DNA, 24 bp, Artificial Sequence
  Description of Artificial Sequence Synthetic oligonucleotide
tgctcttgctactctggcatgtac

SEQ ID NO: 21 - DNA, 23 bp, Artificial Sequence
  Description of Artificial Sequence Synthetic oligonucleotide
aatctacctctgagagctgcagg

SEQ ID NO: 22 - DNA, 23 bp, Artificial Sequence
  Description of Artificial Sequence Synthetic oligonucleotide
tcagacacagctgaagcagaggc

SEQ ID NO: 23 - DNA, 23 bp, Artificial Sequence
  Description of Artificial Sequence Synthetic oligonucleotide
atgccagtgtcagtagatgtcag

SEQ ID NO: 24 - DNA, 24 bp, Artificial Sequence
  Description of Artificial Sequence Synthetic oligonucleotide
tcaagatcagccagagtgcacatg

SEQ ID NO: 25 - DNA, 23 bp, Artificial Sequence
  Description of Artificial Sequence Synthetic oligonucleotide
tgctcttccgagcctctctggag

SEQ ID NO: 26 - DNA, 21 bp, Artificial Sequence
  Description of Artificial Sequence Synthetic oligonucleotide
ctacaatgagctgcgtgtggc

SEQ ID NO: 27 - DNA, 21 bp, Artificial Sequence
  Description of Artificial Sequence Synthetic oligonucleotide
caggtccagacgcaggatggc

SEQ ID NO: 28 - DNA, 23 bp, Artificial Sequence
  Description of Artificial Sequence Synthetic oligonucleotide
gagcacaggactcattcaacagt

SEQ ID NO: 29 - DNA, 26 bp, Artificial Sequence
  Description of Artificial Sequence Synthetic oligonucleotide
tttgtatgtcaacagacagtatccag

SEQ ID NO: 30 - DNA, 20 bp, Artificial Sequence
  Description of Artificial Sequence Synthetic oligonucleotide
tgtgcatacttcgagcggct

SEQ ID NO: 31 - DNA, 20 bp, Artificial Sequence
  Description of Artificial Sequence Synthetic oligonucleotide
ggaaaggcgggagctgatga

SEQ ID NO: 32 - DNA, 21 bp, Artificial Sequence
  Description of Artificial Sequence Synthetic oligonucleotide
tgggaccattcaaaacagcat

SEQ ID NO: 33 - DNA, 21 bp, Artificial Sequence
  Description of Artificial Sequence Synthetic oligonucleotide
gtaaggccaggagaacagatg

SEQ ID NO: 34 - DNA, 19 bp, Artificial Sequence
  Description of Artificial Sequence Synthetic oligonucleotide
tcaaagagaacttcgcggg

SEQ ID NO: 35 - DNA, 22 bp, Artificial Sequence
  Description of Artificial Sequence Synthetic oligonucleotide
cccaagttgtgtccatcctatc

SEQ ID NO: 36 - DNA, 20 bp, Artificial Sequence
  Description of Artificial Sequence Synthetic oligonucleotide
agacaccccattacccaaac

SEQ ID NO: 37 - DNA, 22 bp, Artificial Sequence
  Description of Artificial Sequence Synthetic oligonucleotide
gacaccgcagtaatttcagttg

SEQ ID NO: 38 - DNA, 21 bp, Artificial Sequence
  Description of Artificial Sequence Synthetic oligonucleotide
agcaagacgaaggatgaacag

SEQ ID NO: 39 - DNA, 22 bp, Artificial Sequence
  Description of Artificial Sequence Synthetic oligonucleotide
tttgccctatgattatagccgg

SEQ ID NO: 40 - DNA, 22 bp, Artificial Sequence
  Description of Artificial Sequence Synthetic oligonucleotide
gttgtagataaaggaccctggg

SEQ ID NO: 41 - DNA, 21 bp, Artificial Sequence
  Description of Artificial Sequence Synthetic oligonucleotide
agaaggaaaaggagcagacac

SEQ ID NO: 42 - DNA, 19 bp, Artificial Sequence
  Description of Artificial Sequence Synthetic oligonucleotide
agaaagtggaggtgctgtg

SEQ ID NO: 43 - DNA, 20 bp, Artificial Sequence
  Description of Artificial Sequence Synthetic oligonucleotide
cttctacttcaacgccacca

SEQ ID NO: 44 - DNA, 18 bp, Artificial Sequence
  Description of Artificial Sequence Synthetic oligonucleotide
aagggccggtagaactcc

SEQ ID NO: 45 - DNA, 20 bp, Artificial Sequence
  Description of Artificial Sequence Synthetic oligonucleotide
aacaatgcatccaccagcta

SEQ ID NO: 46 - DNA, 20 bp, Artificial Sequence
  Description of Artificial Sequence Synthetic oligonucleotide
gtaatgacccgcttcttggt

SEQ ID NO: 47 - DNA, 20 bp, Artificial Sequence
  Description of Artificial Sequence Synthetic oligonucleotide
gaagcttaggcccacatctc

SEQ ID NO: 48 - DNA, 20 bp, Artificial Sequence
  Description of Artificial Sequence Synthetic oligonucleotide
ctggaagggtttgaccagag

SEQ ID NO: 49 - DNA, 20 bp, Artificial Sequence
  Description of Artificial Sequence Synthetic oligonucleotide
gcatgtatgaagccgaaatg

SEQ ID NO: 50 - DNA, 20 bp, Artificial Sequence
  Description of Artificial Sequence Synthetic oligonucleotide
tgaactgctccacttccttg

SEQ ID NO: 51 - DNA, 20 bp, Artificial Sequence
  Description of Artificial Sequence Synthetic oligonucleotide
gcctctctcaaggatgaggt

SEQ ID NO: 52 - DNA, 20 bp, Artificial Sequence
  Description of Artificial Sequence Synthetic oligonucleotide
caacaaatgccttgaacctg

SEQ ID NO: 53 - DNA, 21 bp, Artificial Sequence
  Description of Artificial Sequence Synthetic oligonucleotide
ctacaatgagctgcgtgtggc

SEQ ID NO: 54 - DNA, 21 bp, Artificial Sequence
  Description of Artificial Sequence Synthetic oligonucleotide
caggtccagacgcaggatggc

SEQ ID NO: 55 - DNA, 25 bp, Artificial Sequence
  Description of Artificial Sequence Synthetic oligonucleotide
caccgatcagatatccactgacctt

SEQ ID NO: 56 - DNA, 25 bp, Artificial Sequence
  Description of Artificial Sequence Synthetic oligonucleotide
aaacaaggtcagtggatatctgatc

SEQ ID NO: 57 - DNA, 25 bp, Artificial Sequence
  Description of Artificial Sequence Synthetic oligonucleotide
caccgcagcagttcttgaagtactc

SEQ ID NO: 58 - DNA, 25 bp, Artificial Sequence
  Description of Artificial Sequence Synthetic oligonucleotide
aaacgagtacttcaagaactgctgc

SEQ ID NO: 59 - DNA, 38 bp, Artificial Sequence
  Description of Artificial Sequence Synthetic oligonucleotide
tatgggcccacgcgtgagggcctatttcccatgattcc

SEQ ID NO: 60 - DNA, 38 bp, Artificial Sequence
  Description of Artificial Sequence Synthetic oligonucleotide
tgtggatcctcgaggcgggccatttaccgtaagttatg

SEQ ID NO: 61 - DNA, 25 bp, Artificial Sequence
  Description of Artificial Sequence Synthetic oligonucleotide
catgttttcagcattatcagaagga

SEQ ID NO: 62 - DNA, 19 bp, Artificial Sequence
  Description of Artificial Sequence Synthetic oligonucleotide
tgcttgatgtccccccact

SEQ ID NO: 63 - DNA, 23 bp, Artificial Sequence
  Description of Artificial Sequence Synthetic oligonucleotide
ccaccccacaagatttaaacacc

SEQ ID NO: 64 - DNA, 20 bp, Artificial Sequence
  Description of Artificial Sequence Synthetic oligonucleotide
cccttggacccagaggttct

SEQ ID NO: 65 - DNA, 20 bp, Artificial Sequence
  Description of Artificial Sequence Synthetic oligonucleotide
cgagcactttcttgccatga

SEQ ID NO: 66 - DNA, 41 bp, Artificial Sequence
  Description of Artificial Sequence Synthetic oligonucleotide
gcgagcatctgtccactcctgatgctgttatgggcgctcgc

SEQ ID NO: 67 - DNA, 33 bp, Artificial Sequence
  Description of Artificial Sequence Synthetic oligonucleotide
ccggagtacttcaagaactgctgacatcgagct

SEQ ID NO: 68 - DNA, 21 bp, Artificial Sequence
  Description of Artificial Sequence Synthetic oligonucleotide
gcccgagagctgcatccggag

SEQ ID NO: 69 - DNA, 7 bp, Artificial Sequence
  Description of Artificial Sequence Synthetic oligonucleotide
ctaagtt

SEQ ID NO: 70 - DNA, 21 bp, Artificial Sequence
  Description of Artificial Sequence Synthetic oligonucleotide
tacttcaagaactgctgacat

SEQ ID NO: 71 - DNA, 23 bp, Human immunodeficiency virus 1
aaaatctctagcagtggcgcccg

SEQ ID NO: 72 - DNA, 28 bp, Human immunodeficiency virus 1
aaaaggggggactggaagggctaattca

SEQ ID NO: 73 - DNA, 16 bp, Artificial Sequence
  Description of Artificial Sequence Synthetic oligonucleotide
tccggagtacttcaag

SEQ ID NO: 74 - DNA, 30 bp, Artificial Sequence
  Description of Artificial Sequence Synthetic oligonucleotide
gccagggatcagatatccactgacctttgg

SEQ ID NO: 75 - DNA, 25 bp, Homo sapiens
tttaaatgactgtaccaacaactta

SEQ ID NO: 76 - DNA, 25 bp, Homo sapiens
cttaccttgatacttactttgagat

SEQ ID NO: 77 - DNA, 25 bp, Homo sapiens
ttacgtgtgtgccaagtgtggctat

SEQ ID NO: 78 - DNA, 29 bp, Homo sapiens
ctatgagctgttctccagccgctcgaagt

SEQ ID NO: 79 - DNA, 23 bp, Homo sapiens
atcagatatccactgacctttgg

SEQ ID NO: 80 - DNA, 23 bp, Artificial Sequence
  Description of Artificial Sequence Synthetic oligonucleotide
atcatatctacaagggcctttgg

SEQ ID NO: 81 - DNA, 18 bp, Artificial Sequence
  Description of Artificial Sequence Synthetic oligonucleotide
ataatcccagacctttgg

SEQ ID NO: 82 - DNA, 18 bp, Artificial Sequence
  Description of Artificial Sequence Synthetic oligonucleotide
atgtctaaagcccttttg

SEQ ID NO: 83 - DNA, 16 bp, Artificial Sequence
  Description of Artificial Sequence Synthetic oligonucleotide
atctattttcctttgg

SEQ ID NO: 84 - DNA, 15 bp, Artificial Sequence
  Description of Artificial Sequence Synthetic oligonucleotide
tccactggccagtgg

SEQ ID NO: 85 - DNA, 15 bp, Artificial Sequence
  Description of Artificial Sequence Synthetic oligonucleotide
tcaagtcacctttgg

SEQ ID NO: 86 - DNA, 23 bp, Homo sapiens
cagcagttcttgaagtactccgg

SEQ ID NO: 87 - DNA, 22 bp, Artificial Sequence
  Description of Artificial Sequence Synthetic oligonucleotide
cagctactcgggaggcactcgg

SEQ ID NO: 88 - DNA, 19 bp, Artificial Sequence
  Description of Artificial Sequence Synthetic oligonucleotide
agctctttatatactccgg

SEQ ID NO: 89 - DNA, 17 bp, Homo sapiens
ccggagtacttcaagaa

SEQ ID NO: 90 - DNA, 18 bp, Homo sapiens
ccggagttacttcaagaa

SEQ ID NO: 91 - DNA, 15 bp, Homo sapiens
ccactacttcaagaa

SEQ ID NO: 92 - DNA, 284 bp, Artificial Sequence
  Description of Artificial Sequence Synthetic polynucleotide
caccagagcacaggactcattcaacagtcctcaatattcttatgtggttgaagtgtttaatatctaatttaatcataatctgaaatgttcttaaaaagtggttattttttaaatctcaaagtaagtatcaaggtaagtggaagggctaattcactcccaacgaagacaagatatccttgatctgtggatctaccacacacaaggctacttccctgattggcagaactacacaccagggccagggatcagatatccactgacctttggatggtgctacaagctag

SEQ ID NO: 93 - DNA, 230 bp, Artificial Sequence
  Description of Artificial Sequence Synthetic polynucleotide
tcaagtagtgtgtgcccgtctgttgtgtgactctggtaactagagatccctcagacccttttagtcagtgtggaaaatctctagcagtaagttgttggtacagtcatttaaatttcagctccattttaaaaaattaattggaggacaaaaatcagcagggaatatttcaagatattttcttttattaactatttagactggatactgtctgttgacatacaaatttgatc

SEQ ID NO: 94 - DNA, 294 bp, Artificial Sequence
  Description of Artificial Sequence Synthetic polynucleotide
cacagcctaccctcggaacgggggcagcgctgtctttgcctgggttggtggatttgggagcttgaccccggaaaggcgggagctgatgactccacatttgcctctccttccaccacaggcgtttacgtgtgtgccaagtgtggctattggaagggctaattcactcccaacgaagacaagatatccttgatctgtggatctaccacacacaaggctacttccctgattggcagaactacacaccagggccagggatcagatatccactgacctttggatggtgctacaagctag

SEQ ID NO: 95 - DNA, 212 bp, Artificial Sequence
  Description of Artificial Sequence Synthetic polynucleotide
aagtagtgtgtgcccgtctgttgtgtgactctggtaactagagatccctcagacccttttagtcagtgtggaaaatctctagcagtgctatgagctgttctccagccgctcgaagtatgcacactcgtctccatggccggcgttcaccgagaccattcacgccgacagcgtggccaagcgtccggagcacaatagatctgaagccttgaagg

SEQ ID NO: 96 - DNA, 635 bp, Artificial Sequence
  Description of Artificial Sequence Synthetic polynucleotide
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcag

SEQ ID NO: 97 - DNA, 900 bp, Artificial Sequence
  Description of Artificial Sequence Synthetic polynucleotide
  modified_base (743)..(743): a, c, t, g, unknown or other
  modified_base (829)..(830): a, c, t, g, unknown or other
  modified_base (894)..(894): a, c, t, g, unknown or other
gagcacaggactcattcaacagtcctcaatattcttatgtggttgaagtgtttaatatctaatttaatcataatctgaaatgttcttaaaaagtggttattttttaaatctcaaagtaagtatcaaggtaagtggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagctaagtttacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcaganccttttagtcagtgtggaaaatctctagcagtaagttgttggtacagtcatttaaatttcagctccattttaaaaaattaattggnngacaaaaatcagcaggaatatttcaagatattttcttttattaactatttagactggatactgnctgttg

SEQ ID NO: 98 - DNA, 634 bp, Artificial Sequence
  Description of Artificial Sequence Synthetic polynucleotide
tggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgcttgagtgcttcaagtagtgtgtgcccgtctgttgtgtgactctggtaactagagatccctcagacccttttagtcagtgtggaaaatctctagcac

SEQ ID NO: 99 - DNA, 50 bp, Artificial Sequence
  Description of Artificial Sequence Synthetic oligonucleotide
gcatccggagctaagtttacttcaagaactgctgacatcgagcttgctac

SEQ ID NO: 100 - DNA, 760 bp, Artificial Sequence
  Description of Artificial Sequence Synthetic polynucleotide
ggaaaggcgggagctgatgactccacatttgcctctccttccaccacaggcgtttacgtgtgtgccaagtgtggctattggaagggctaattcactcccaacgaagacaagatatccttgatctgtggatctaccacacacaaggctacttccctgattggcagaactacacaccagggccagggatcagatatccactgacgggttattctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagggatacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagctatgagctgttctccagccgctcgaagtatgcaca

SEQ ID NO: 101 - DNA, 53 bp, Artificial Sequence
  Description of Artificial Sequence Synthetic oligonucleotide
accagggccagggatcagatatccactgacgggttattctttggatggtgcta

SEQ ID NO: 102 - DNA, 53 bp, Artificial Sequence
  Description of Artificial Sequence Synthetic oligonucleotide
tgcatccggagggatacttcaagaactgctgacatcgagcttgctacaaggga